(19) United States
(12) Patent Application Publication
(10) Pub. No.: US 2025/0312251 A1
(43) Pub. Date: Oct. 9, 2025

(54) COMPOSITIONS AND METHODS FOR ALTERING THE COLOR OF HAIR

(71) Applicant: L'OREAL, Paris (FR)

(72) Inventors: Sarah Mary SPEER, Linden, NJ (US); Tathagata MUKHERJEE, Union, NJ (US); Shahid Mohammad NASEER, Hasbrouck Heights, NJ (US)

(21) Appl. No.: 18/626,281

(22) Filed: Apr. 3, 2024

(51) Int. Cl. A61K 8/41; A61K 8/365; A61K 8/46; A61Q 5/00

(52) U.S. Cl. CPC A61K 8/41; A61K 8/365; A61K 8/46; A61Q 5/00; A61K 2800/884

(57) ABSTRACT

The disclosure relates to compositions and methods for altering the color of hair, the compositions comprising at least one organic amine and at least one solvent. The compositions are used as pre- and/or post-treatment compositions in methods according to the disclosure.

### TECHNICAL FIELD

The disclosure relates to compositions and methods for altering the color, tone, and/or shade of hair. The compositions and methods can be used to alter the color of hair by removing various color, tones, and/or shades from the hair.

### BACKGROUND

Consumers often desire to change the color of their hair. For example, as a person ages, the hair follicle loses its natural pigment, resulting in grey, silver, or white hair. It is common for consumers to color their grey hairs, for example to match their natural hair color. Similarly, consumers may wish to change the color, tone, and/or shade of their hair from that of their natural color or a previous hair dye, e.g. may wish change to a different color entirely, may wish to lighten or darken their hair color, may wish to add warmth or coolness to the color of their hair, etc. The most common hair dyeing processes are permanent, semi-permanent, and temporary hair dyeing.

Semi-permanent or temporary hair dyeing compositions typically use pigments, liposoluble dyes, or direct dyes which are deposited onto the hair fiber to impart color to the hair. Generally, these hair dyes are washed out of the hair after one to several shampoo cycles.

On the other hand, permanent hair dyeing compositions uses oxidation dye precursors, which are also known as primary intermediates or couplers. In order to provide various colors, tones, and shades of permanent hair color, dye compositions contain various combinations of different types of oxidation dye compounds. These oxidation dye compounds are small, colorless or weakly colored compounds which, when combined with oxidizing agents, give rise to colored complexes by a process of oxidative condensation. The permanent hair dye compositions also contain ammonia or other alkalizing agents which causes the hair shaft to swell, thus allowing the oxidative dye molecules to penetrate the cuticle and cortex before the oxidation condensation process is completed. The resulting larger-sized colored complexes from the oxidative reaction are then trapped inside the hair fiber and cannot be washed out, thereby permanently altering the color of the hair.

Because permanent hair dyes are not able to be washed out of the hair, if an individual who has previously changed the color or tone of their hair with a first oxidative dye composition wishes to alter the color or tone of their previously-colored hair, some or all of the color imparted by the first oxidative dye composition must be neutralized. For example, a consumer may wish to remove the first color or tone in order to go back to their natural hair color, or to achieve a second color different from the first color by using a second oxidative dye composition. In such cases it is necessary to remove all of the first color from the hair, either to allow the natural hair color to be visible or to prevent the first color from interfering with the appearance of the second color. As another example, a consumer may wish to achieve a second tone or shade in the same color family as the first color, such as changing hair color from dark brown to light brown, and therefore may desire to remove some, but not necessarily all, of the first color. Alternatively, the change desired may be more subtle, for example by increasing the warmth or brightness of the first color. Thus, depending on the particular combination of oxidation dye compounds present in the first dye composition, and the desired second color or tone to be achieved, different levels and types of color removal are required.

Although there are known methods that are effective for removing the first color from the hair, these methods have drawbacks. For example, one common color-removal process uses oxidizing agents, but these compositions are harsh and lead to damage to the hair, causing the hair to be weak, brittle, and have an unhealthy appearance. This can be particularly problematic since the hair has already been subjected to harsh chemicals such as alkalizing agents and oxidizing agents in the first oxidative dye composition, and the integrity of the hair fibers is therefore already compromised. Moreover, these compositions can remove essentially all color from the hair, and therefore subsequent application of a second oxidative dye composition is needed to achieve the desired second color or tone. This step, known as “backfilling” the color, further exposes the hair to harsh chemicals and leads to additional damage.

In view of the damage associated with the use of oxidizing agents as color removers, a second option involves neutralizing the first oxidative dye molecules with color-removing agents such as thiol-based compounds. However, these compositions have not been as effective at removing color as those using oxidizing agents. For example, some oxidative dye compounds, particularly those imparting darker colors such as blacks, ash blacks, ash browns, browns, ash mochas, natural ash (blue or green), ash violets, violets, red violets, reds, red mochas, red browns, mahogany, red coppers, coppers, and golds, are more resistant to removal using thiol-based color-removing agents than to removal using oxidizing agents. As such, the color result achieved with these color-removing agents may not be satisfactory when used on hair that has been dyed with such compounds, and the remaining color will interfere with the colorist's attempt to achieve the desired color, tone, or shade.

Although attempts to solve these problems have been made, for example by adding components to the composition containing the oxidizing agent to minimize hair damage or by using different color-removing agents, to date there has not been a composition or process that is effective at removing oxidative color to permit a change in hair color, that adequately minimizes or eliminates damage to the hair. Thus, the choices that are available today for consumers who wish to alter the color of their previously-dyed hair are to either (1) remove all hair color with a color-removing composition containing damaging oxidizing agents, and subsequently expose the hair to further damage with a second oxidative dye composition to achieve the desired color or tone, or (2) remove only the hair dyes from the first color that are susceptible to removal with other color-removing agents such as thiol-based compounds, and attempt to achieve the desired color or tone with a second oxidative dye composition despite the remaining first color.

It has now been surprisingly discovered that methods of treating hair with pre- and/or post-treatment compositions permit controlled removal of artificial color from previously-dyed hair, providing desired alterations in hair color, tone, and/or shade. The compositions are unique in that they can thus be used in color-removal processes to achieve a desired color, tone, and/or shade, much like consumers experience when undergoing a hair dye process, even in the absence of a backfilling step. Further, the methods surprisingly increase the overall color-removal performance of the hair color-removing compositions. Finally, because the compositions are free or substantially free of oxidizing agents, damage to hair typically associated with hair color-removing compositions and processes can be minimized or eliminated.

### SUMMARY

The disclosure relates to compositions and methods that can be used to remove artificial color from the hair, in particular hair that has previously been dyed with an oxidative dye composition. In various embodiments, the compositions and methods can remove some or all of certain dyes, while having minimal or no impact on other dyes. As such, the color left on the hair can be “tuned” to achieve the consumer's desired result. The compositions and methods are able to achieve a desired hair color, tone, and/or shade even without, or with minimal, application of, subsequent hair coloring compositions.

In various embodiments, the disclosure relates to compositions for use as pre- and/or post-treatment compositions in processes for treating keratin fibers such as hair, for example for removing color from the hair, the treatment compositions comprising (a) at least one organic amine, and (b) at least one solvent. The treatment compositions optionally comprise at least one additional compound chosen from surfactants, fatty compounds, thickening agents, amino acids and/or salts thereof, amino-sulfonic acids and/or salts thereof, organic amine compounds, or combinations of two or more thereof. The solvent may be chosen from water and/or non-aqueous solvents. The pH of the compositions is generally in the range of about 9 to about 13, such as about 9.5 to about 12.5, or about 10 to about 12.

In various embodiments, the organic amine is chosen from alkanolamines, such as monoethanolamine, diethanolamine, or triethanolamine, or may be aminomethyl propanol. The total amount of organic amines in the treatment composition can range from about 0.1% up to about 25%, such as from about 1% to about 25%, from about 5% to about 25%, or from 5% to about 20% by weight, relative to the total weight of the composition.

In various embodiments, the treatment compositions comprise one or more additional components chosen from surfactants, fatty compounds, thickening agents, amino acids and/or salts thereof, amino-sulfonic acids and/or salts thereof, or combinations of two or more thereof.

The treatment compositions can be used in processes for removing color from hair and/or altering the color, tone, and or shade of the hair, when used in processes as pre- and/or post-treatments with hair color-removing compositions. The hair color-removing compositions can include traditional hair color-removing agents, for example thiol-based compounds such as thiolactic acid, thiosalicylic acid, and/or salts thereof, and/or keto-glutaric acid and/or salts thereof. In preferred embodiments, the hair color-altering compositions do not include oxidizing agents. The hair color-removing compositions may optionally include at least one additional compound chosen from clays, surfactants, fatty compounds, thickening agents, amino acids and/or salts thereof, amino-sulfonic acids and/or salts thereof, organic amine compounds, or combinations of two or more thereof.

The disclosure also relates to kits. The kits may include a first compartment or container comprising a treatment composition according to the disclosure, and a second compartment or container comprising a hair color-removing composition.

In further embodiments, the disclosure relates to methods of treating hair using treatment compositions according to the disclosure. For example, the methods may be methods of altering the color of hair, in particular by removing some or all of certain dyes. In some embodiments, the compositions remove some or all of certain dyes while having minimal or no impact on other dyes, thus permitting a controlled alteration of the hair color. Although some methods comprise subsequent application of a hair coloring composition, the compositions and methods described herein permit desired results to be achieved even in the absence of such subsequent coloring process.

In various embodiments, a treatment composition according to the disclosure may be applied to the hair as a pre-treatment, which may be applied onto keratin fibers, e.g. hair that has been previously-dyed with at least one oxidative dye compound, and optionally left on the keratin fibers for a period of time, and subsequently a hair color-removing composition can be applied onto the keratin fibers, and optionally left on the keratin fibers for a period of time. In other embodiments, a treatment composition according to the disclosure may be applied to the hair as a post-treatment, which may be applied onto keratin fibers and optionally left on the keratin fibers for a period of time after a hair color-removing composition has been applied onto the keratin fibers and optionally left on the keratin fibers for a period of time. In various embodiments the leave-in period of time may range from about 1 minute to about 1 hour, from about 1 minute to about 45 minutes, from about 1 minute to about 30 minutes, from about 5 minutes to about 1 hour, from about 5 minutes to about 45 minutes, from about 5 minutes to about 30 minutes, etc.

### DETAILED DESCRIPTION

The disclosure relates to compositions and methods for altering the color of the hair, in particular hair that has been previously dyed with an oxidative dye composition. The methods comprise removing some or all of various dye compounds in order to provide a desired color, tone, and/or shade of the treated hair.

### I. Compositions

Compositions according to the disclosure (“treatment compositions”) comprise (a) at least one organic amine compound, and (b) at least one solvent. The compositions are used as pre- and/or post-treatment compositions in processes for removing hair color and/or altering the color of hair.

### A. Treatment Compositions

### Organic Amine Compounds

The treatment compositions according to the disclosure comprise at least one organic amine compound. Optionally, the compositions may comprise more than one organic amine compound, such as at least two organic amine compounds.

In various embodiments, alkanolamines, such as mono-, di-, or trialkanolamines, comprising from one to three identical or different C1-C4 hydroxyalkyl radicals, may be used. By way of example, organic amines such as monoethanolamine, diethanolamine, triethanolamine, monoisopropanolamine, diisopropanolamine, triisopropanolamine, N,N-dimethylethanolamine, 2-amino-2-methyl-1-propanol (AMP), 2-amino-2-methyl-1,3-propanediol, 3-amino-1,2-propanediol, 3-dimethylamino-1,2-propanediol, tris(hydroxymethyl) amino methane, guanidine, or combinations of two or more thereof may be chosen.

The total amount of organic amine compounds may range from about 0.1% up to about 25%, such as up to about 20%, up to about 18%, or up to about 15% by weight, relative to the total weight of the composition. For example, the total amount of organic amine compounds may range from about 1% to about 20%, from about 3% to about 18%, or from about 5% to about 15% by weight, relative to the total weight of the composition. In at least some embodiments, the compositions have a total amount of organic amine compounds ranging from about 1% to about 20%, from about 1% to about 18%, from about 1% to about 15%, from about 1% to about 14%, from about 1% to about 13%, from about 1% to about 12%, from about 1% to about 11%, from about 1% to about 10%, from about 1% to about 9%, from about 1% to about 8%, from about 1% to about 7%, from about 1% to about 6%, from about 1% to about 5%, from about 3% to about 20%, from about 3% to about 18%, from about 3% to about 15%, from about 3% to about 14%, from about 3% to about 13%, from about 3% to about 12%, from about 3% to about 11%, from about 3% to about 10%, from about 3% to about 9%, from about 3% to about 8%, from about 3% to about 7%, from about 3% to about 6%, from about 3% to about 5%, from about 5% to about 20%, from about 5% to about 18%, from about 5% to about 15%, from about 5% to about 14%, from about 5% to about 13%, from about 5% to about 12%, from about 5% to about 11%, from about 5% to about 10%, from about 5% to about 9%, from about 5% to about 8%, from about 5% to about 7%, from about 5% to about 6%, from about 7% to about 20%, from about 7% to about 18%, from about 7% to about 15%, from about 7% to about 14%, from about 7% to about 13%, from about 7% to about 12%, from about 7% to about 11%, from about 7% to about 10%, from about 7% to about 9%, from about 7% to about 8%, from about 9% to about 20%, from about 9% to about 18%, from about 9% to about 15%, from about 9% to about 14%, from about 9% to about 13%, from about 9% to about 12%, from about 9% to about 11%, or from about 9% to about 10% by weight, relative to the total weight of the composition. In various embodiments, the total amount of organic amine compounds may be about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, or about 15% by weight, relative to the total weight of the composition, or may be present in any range using any of the foregoing as upper and lower limits.

### Solvents

The treatment compositions according to the disclosure comprise at least one solvent. The solvent may be chosen from water, non-aqueous solvents, or combinations thereof.

In some embodiments, the solvent comprises, consists essentially of, or consists of water. The total amount of water may vary depending on the desired properties of composition, for example consistency, viscosity, etc.

In some embodiments, non-aqueous solvents may be used, for example, glycerin, C1-4 alcohols, fatty alcohols, fatty ethers, fatty esters, polyols, glycols, vegetable oils, mineral oils, liposomes, laminar lipid materials, or combinations thereof. Non-limiting examples of non-aqueous solvents include alkanediols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or combinations thereof.

The total amount of solvents in the composition typically ranges from about 50% to about 98%, such as from about 60% to about 98%, from about 70% to about 98%, from about 75% to about 98%, from about 75% to about 95%, or from about 80% to about 90% by weight, relative to the total weight of the composition.

### Auxiliary Components

The treatment compositions according to the disclosure may optionally include one or more auxiliary components. Non-limiting examples include surfactants, amino acids, preservatives, fragrances such as parfum, pH adjusters, salts, antioxidants, vitamins (e.g. ascorbic acid, tocopherol), vitamin derivatives, botanical extracts, buffers, sequestering agents, dyes or pigments for coloring the composition, and the like. In some embodiments, the compositions are free or essentially free of any of the aforementioned, e.g. the compositions may be free or essentially free of antioxidants, etc.

The total amount of auxiliary components, if present, typically ranges from about 0.01% to about 15% by weight, based on the total weight of the composition. For example, in some embodiments the individual amounts of each component or the total amount of components may range from about 0.1% to about 10%, about 0.1% to about 8%, about 0.1% to about 5%, about 0.1% to about 4%, about 0.1% to about 3%, about 0.1% to about 2%, about 0.25% to about 10%, about 0.25% to about 8%, about 0.25% to about 5%, about 0.25% to about 4%, about 0.25% to about 3%, about 0.25% to about 2%, about 0.5% to about 10%, about 0.5% to about 8%, about 0.5% to about 5%, about 0.5% to about 4%, about 0.5% to about 3%, about 0.5% to about 2%, about 0.75% to about 10%, about 0.75% to about 8%, about 0.75% to about 5%, about 0.75% to about 4%, about 0.75% to about 3%, or about 0.75% to about 2% by weight, based on the total weight of the composition.

The treatment compositions may be in any suitable form. For example, the compositions may be a liquid, a gel, a gel cream, a cream, a serum, etc.

The pH of the treatment composition is typically alkaline, for example ranging from about 9 to about 13. In some embodiments, the treatment compositions have a pH ranging from about 9.5 to about 12.5, from about 10 to about 12.5, from about 10.5 to about 12.5, or from about 11 to about 12.

### B. Hair Color Removing Compositions

Hair color-removing compositions that can be used in conjunction with the treatment compositions according to the disclosure are not limited. In various embodiments, the hair color-removing compositions comprise at least one hair color-removing agent and at least one solvent, and optionally may further include at least one additional compound chosen from clays, surfactants, fatty compounds, thickening agents, organic acids and/or salts thereof, amino acids and/or salts thereof, amino-sulfonic acids and/or salts thereof, organic amine compounds, or combinations of two or more thereof.

### Hair Color-Removing Agents

Compositions according to the disclosure comprise at least one hair color-removing agent. Color-removing agents according to the disclosure are preferably not oxidizing agents. By way of example, useful color-removing agents may be chosen from thiosalicylic acid and/or salts thereof, thiolactic acid and/or salts thereof, thioglycolic acid and/or esters thereof, glutaric acid and/or salts thereof, glyoxylic acid and/or salts thereof, citric acid and/or salts thereof, cysteine and/or salts thereof, homocysteine and/or salts thereof, borohydrides and/or derivatives thereof, phosphines and/or salts thereof, bisulphites and/or salts thereof, sulphites and/or salts thereof, or combinations of two or more thereof. Non-limiting examples of salts include sodium salts, ammonium salts, lithium salts, potassium salts, and calcium salts.

In some embodiments, the color-removing agents comprise, consist essentially of, or consist of thiol-based compounds. Exemplary thiol-based color-removing agents include thiolactic acid and/or salts thereof, thioglycolic acid and/or esters thereof, thiosalicylic acid and/or salts thereof, or combinations of two or more thereof.

In various embodiments, the total amount of color-removing agents may range from about 0.5% to about 20%, such as from about 1% to about 18%, from 2% to about 15%, or from about 4% to about 12% by weight, relative to the total weight of the composition. For example, in various embodiments, the total amount of color-removing agents may range from about 1% to about 20%, from about 1% to about 15%, from about 1% to about 14%, from about 1% to about 13%, from about 1% to about 12%, from about 1% to about 11%, from about 1% to about 10%, from about 1% to about 9%, from about 1% to about 8%, from about 1% to about 7%, from about 1% to about 6%, from about 1% to about 5%, from about 2% to about 20%, from about 2% to about 15%, from about 2% to about 14%, from about 2% to about 13%, from about 2% to about 12%, from about 2% to about 11%, from about 2% to about 10%, from about 2% to about 9%, from about 2% to about 8%, from about 2% to about 7%, from about 2% to about 6%, from about 2% to about 5%, from about 3% to about 20%, from about 3% to about 15%, from about 3% to about 14%, from about 3% to about 13%, from about 3% to about 12%, from about 3% to about 11%, from about 3% to about 10%, from about 3% to about 9%, from about 3% to about 8%, from about 3% to about 7%, from about 3% to about 6%, from about 3% to about 5%, from about 4% to about 20%, from about 4% to about 15%, from about 4% to about 14%, from about 4% to about 13%, from about 4% to about 12%, from about 4% to about 11%, from about 4% to about 10%, from about 4% to about 9%, from about 4% to about 8%, from about 4% to about 7%, from about 4% to about 6%, from about 4% to about 5%, from about 5% to about 20%, from about 5% to about 15%, from about 5% to about 14%, from about 5% to about 13%, from about 5% to about 12%, from about 5% to about 11%, from about 5% to about 10%, from about 5% to about 9%, from about 5% to about 8%, from about 5% to about 7%, or from about 5% to about 6%, from about 6% to about 20%, from about 6% to about 15%, from about 6% to about 14%, from about 6% to about 13%, from about 6% to about 12%, from about 6% to about 11%, from about 6% to about 10%, from about 6% to about 9%, from about 6% to about 8%, from about 6% to about 7%, from about 7% to about 20%, from about 7% to about 15%, from about 7% to about 14%, from about 7% to about 13%, from about 7% to about 12%, from about 7% to about 11%, from about 7% to about 10%, from about 7% to about 9%, from about 7% to about 8%, from about 8% to about 20%, from about 8% to about 15%, from about 8% to about 14%, from about 8% to about 13%, from about 8% to about 12%, from about 8% to about 11%, from about 8% to about 10%, from about 8% to about 9%, from about 9% to about 20%, from about 9% to about 15%, from about 9% to about 14%, from about 9% to about 13%, from about 9% to about 12%, from about 9% to about 11%, from about 9% to about 10%, from about 10% to about 20%, from about 10% to about 15%, from about 10% to about 14%, from about 10% to about 13%, from about 10% to about 12%, or from about 10% to about 11% by weight, relative to the total weight of the composition. For example, the total amount of color-removing agents may be about 1%, about 2%, about 3%, about 4%, about 5%, about 6%, about 7%, about 8%, about 9%, about 10%, about 11%, about 12%, about 13%, about 14%, or about 15% by weight, relative to the total weight of the composition, including any range using any of the foregoing as upper and lower limits.

In some embodiments, the hair color-removing compositions comprise a total amount of thiol-based color-removing agents in any of the foregoing ranges or amounts. For example, the compositions may comprise one or more of thiosalicylic acid, thiolactic acid, and/or salts thereof as color-removing agents, and the total amount of thiosalicylic acid, thiolactic acid, and salts thereof may be at least about 1%, for example may range from about 1% to about 20%, from about 1% to about 18%, from about 1% to about 15%, from about 1% to about 12%, from about 1% to about 10%, from about 1% to about 8%, from about 1% to about 6%, from about 1.5% to about 20%, from about 1.5% to about 18%, from about 1.5% to about 15%, from about 1.5% to about 12%, from about 1.5% to about 10%, from about 1.5% to about 8%, from about 1.5% to about 6%, from about 2% to about 20%, from about 2% to about 18%, from about 2% to about 15%, from about 2% to about 12%, from about 2% to about 10%, from about 2% to about 8%, from about 2% to about 6%, from about 2.5% to about 20%, from about 2.5% to about 18%, from about 2.5% to about 15%, from about 2.5% to about 12%, from about 2.5% to about 10%, from about 2.5% to about 8%, from about 2.5% to about 6%, from about 3% to about 20%, from about 3% to about 18%, from about 3% to about 15%, from about 3% to about 12%, from about 3% to about 10%, from about 3% to about 8%, from about 3% to about 6%, from about 3.5% to about 20%, from about 3.5% to about 18%, from about 3.5% to about 15%, from about 3.5% to about 12%, from about 3.5% to about 10%, from about 3.5% to about 8%, from about 3.5% to about 6%, from about 4% to about 20%, from about 4% to about 18%, from about 4% to about 15%, from about 4% to about 12%, from about 4% to about 10%, from about 4% to about 8%, from about 4% to about 6%, from about 4.5% to about 20%, from about 4.5% to about 18%, from about 4.5% to about 15%, from about 4.5% to about 12%, from about 4.5% to about 10%, from about 4.5% to about 8%, or from about 4.5% to about 6% by weight, relative to the total weight of the composition. For example, the composition may comprise a total amount of thiol-based color-removing agents, e.g. a total among of thiosalicylic acid, thiolactic acid, and salts thereof, of about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight, relative to the total weight of the composition, or may be present in a range using any of the foregoing as upper or lower limits. In such embodiments, the compositions may optionally comprise at least one additional color-removing agent in addition to thiol-based agents, e.g. in addition to thiosalicylic acid, thiolactic acid, and/or salts thereof.

### Solvents

Hair color-removing compositions that can be used in methods according to the disclosure comprise at least one solvent. The solvent may be chosen from water, non-aqueous solvents, or combinations thereof.

In some embodiments, the solvent comprises, consists essentially of, or consists of water. The total amount of water may vary depending on the desired properties of composition, for example consistency, viscosity, etc.

In some embodiments, non-aqueous solvents may be used, for example, glycerin, C1-4 alcohols, fatty alcohols, fatty ethers, fatty esters, polyols, glycols, vegetable oils, mineral oils, liposomes, laminar lipid materials, or combinations thereof. Non-limiting examples of non-aqueous solvents include alkanediols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, caprylyl glycol, 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, or combinations thereof.

The total amount of solvents in the hair-color removing composition typically ranges from about 50% to about 98%, such as from about 60% to about 98%, from about 70% to about 98%, from about 75% to about 98%, from about 75% to about 95%, or from about 80% to about 90% by weight, relative to the total weight of the composition.

### Organic Acids

Hair color-removing compositions that can be used optionally comprise at least one organic acid, for example chosen from carboxylic acids, which includes, for example, α-hydroxy acids and/or keto acids. Salts of organic acids may also be chosen and are expressly included unless stated otherwise.

In various embodiments, useful organic acids include citric acid, maleic acid, succinic acid, aspartic acid, glutamic acid, lactic acid, malic acid, tartaric acid, glyceric acid, gluconic acid, glutaric acid, acetic acid, glycolic acid, oxalic acid, 2-ketobutyric acid, β-hydroxypyruvic acid, ketomalonic acid, oxoacetic acid, 2-ketoglutaric acid, 2-keto-L-gulonic acid, 2-ketoglutaric acid dihydrate, pyruvamide salts thereof, or combinations thereof. In some embodiments, the composition comprises one or more organic acids chosen from 2-oxoglutaric acid (ketoglutaric acid), glyoxylic acid, glycolic acid, maleic acid, citric acid, lactic acid, acetic acid, salts thereof, or combinations of two or more thereof.

In some embodiments, the hair-color removing compositions comprise at least one carboxylic acid. In some embodiments, the compositions comprise at least one keto acid. Without intending to be bound by theory, it is believed that keto acids, which are acids containing two oxygen-containing functional groups, a carboxylic acid (—COOH) and a ketone group (>C═O), increase color-removal benefits of color-removing agents.

If present, the total amount of organic acids and salts thereof may range from about 0.01% up to about 40%, such as up to about 38%, up to about 35%, up to about 30%, up to about 28%, up to about 25%, up to about 20%, up to about 15%, up to about 13%, up to about 10%, up to about 9%, up to about 8%, up to about 7%, up to about 6%, or up to about 5% by weight, relative to the total weight of the hair-color removing composition.

For example, the hair-color removing compositions may comprise at least one carboxylic acid and the total amount of carboxylic acids may range from about 0.1% to about 30%, from about 0.1% to about 28%, from about 0.1% to about 25%, from about 0.1% to about 20%, from about 0.1% to about 15%, from about 0.1% to about 13%, from about 0.1% to about 10%, from about 0.1% to about 8%, from about 0.1% to about 6%, from about 0.1% to about 5%, from about 0.5% to about 30%, from about 0.5% to about 28%, from about 0.5% to about 25%, from about 0.5% to about 20%, from about 0.5% to about 15%, from about 0.5% to about 13%, from about 0.5% to about 10%, from about 0.5% to about 8%, from about 0.5% to about 6%, from about 0.5% to about 5%, from about 1% to about 30%, from about 1% to about 28%, from about 1% to about 25%, from about 1% to about 20%, from about 1% to about 15%, from about 1% to about 13%, from about 1% to about 10%, from about 1% to about 8%, from about 1% to about 6%, from about 1% to about 5%, from about 1.5% to about 30%, from about 1.5% to about 28%, from about 1.5% to about 25%, from about 1.5% to about 20%, from about 1.5% to about 15%, from about 1.5% to about 13%, from about 1.5% to about 10%, from about 1.5% to about 8%, from about 1.5% to about 6%, from about 1.5% to about 5%, from about 2% to about 30%, from about 2% to about 28%, from about 2% to about 25%, from about 2% to about 20%, from about 2% to about 15%, from about 2% to about 13%, from about 2% to about 10%, from about 2% to about 8%, from about 2% to about 6%, from about 2% to about 5%, from about 3% to about 30%, from about 3% to about 28%, from about 3% to about 25%, from about 3% to about 20%, from about 3% to about 15%, from about 3% to about 13%, from about 3% to about 10%, from about 3% to about 8%, from about 3% to about 6%, from about 3% to about 5%, from about 4% to about 30%, from about 4% to about 28%, from about 4% to about 25%, from about 4% to about 20%, from about 4% to about 15%, from about 4% to about 13%, from about 4% to about 10%, from about 4% to about 8%, from about 5% to about 30%, from about 5% to about 28%, from about 5% to about 25%, from about 5% to about 20%, from about 5% to about 15%, from about 5% to about 13%, from about 5% to about 10%, from about 5% to about 8%, from about 6% to about 30%, from about 6% to about 28%, from about 6% to about 25%, from about 6% to about 20%, from about 6% to about 15%, from about 6% to about 13%, from about 6% to about 10%, from about 6% to about 8%, from about 8% to about 30%, from about 8% to about 28%, from about 8% to about 25%, from about 8% to about 20%, from about 8% to about 15%, from about 8% to about 13%, from about 10% to about 30%, from about 10% to about 28%, from about 10% to about 25%, from about 10% to about 20%, from about 10% to about 15%, from about 10% to about 13%, from about 12% to about 30%, from about 12% to about 28%, from about 12% to about 25%, from about 12% to about 20%, from about 12% to about 15%, or from about 12% to about 13% by weight, relative to the total weight of the composition. In some embodiments, the total amount of carboxylic acids may be about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 21%, about 22%, about 23%, about 24%, or about 25% by weight, relative to the total weight of the hair-color removing composition, including all ranges and subranges using any of the foregoing as upper and lower limits.

In other embodiments, the hair-color removing compositions comprise at least one keto acid, and the total amount of keto acids ranges from about 0.01% up to about 20%, up to about 15%, up to about 13%, up to about 10%, up to about 9%, up to about 8%, up to about 7%, up to about 6%, or up to about 5% by weight, relative to the total weight of the composition. For example, the total amount of keto acids may range from about 0.1% to about 20%, from about 0.1% to about 15%, from about 0.1% to about 13%, from about 0.1% to about 10%, from about 0.1% to about 8%, from about 0.5% to about 20%, from about 0.5% to about 15%, from about 0.5% to about 13%, from about 0.5% to about 10%, from about 0.5% to about 8%, from about 1% to about 20%, from about 1% to about 15%, from about 1% to about 13%, from about 1% to about 10%, from about 1% to about 8%, from about 1.5% to about 20%, from about 1.5% to about 15%, from about 1.5% to about 13%, from about 1.5% to about 10%, from about 1.5% to about 8%, from about 2% to about 20%, from about 2% to about 15%, from about 2% to about 13%, from about 2% to about 10%, from about 2% to about 8%, from about 3% to about 20%, from about 3% to about 15%, from about 3% to about 13%, from about 3% to about 10%, from about 3% to about 8%, from about 4% to about 20%, from about 4% to about 15%, from about 4% to about 13%, from about 4% to about 10%, from about 4% to about 8%, from about 5% to about 20%, from about 5% to about 15%, from about 5% to about 13%, from about 5% to about 10%, from about 5% to about 8%, from about 6% to about 20%, from about 6% to about 15%, from about 6% to about 13%, from about 6% to about 10%, from about 6% to about 8%, from about 8% to about 20%, from about 8% to about 15%, from about 8% to about 13%, from about 8% to about 12%, from about 8% to about 11%, from about 9% to about 20%, from about 9% to about 15%, from about 9% to about 13%, from about 9% to about 12%, or from about 9% to about 11% by weight, relative to the total weight of the composition. In some embodiments, the total amount of keto acids may be about 5%, about 5.5%, about 6%, about 6.5%, about 7%, about 7.5%, about 8%, about 8.5%, about 9%, about 9.5%, about 10%, about 10.5%, about 11%, about 11.5%, about 12%, about 12.5%, about 13%, about 13.5%, about 14%, about 14.5%, or about 15% by weight, relative to the total weight of the composition, including all ranges and subranges using any of the foregoing as upper and lower limits.

It may, in some embodiments, be advantageous to include at least one keto acid in the hair-color removing composition in an amount relative to the color-removing agent to maximize the color-removal benefits. Thus, in various embodiments, the compositions comprise at least one keto acid where the weight ratio of total amounts of color-removing agent(s): keto acid(s) ranges from about 1:0.5 to 1:10. In other embodiments, it may be advantageous for the weight ratio of total amounts of color-removing agent(s): keto acid(s) to be equal to or less than 1. For example, the weight ratio of total amounts of color-removing agent(s): keto acid(s) may range from about 1:1 to about 1:10, such as from about 1:1 to about 1:8, from about 1:1 to about 1:6, from about 1:1 to about 1:5, from about 1:1 to about 1:4, from about 1:1 to about 1:3, from about 1:1 to about 1:2, from about 1:1 to about 1:1.5, from about 1:1.1 to about 1:10, about 1:1.1 to about 1:8, from about 1:1.1 to about 1:6, from about 1:1.1 to about 1:5, from about 1:1.1 to about 1:4, from about 1:1.1 to about 1:3, from about 1:1.1 to about 1:2, or from about 1:1.1 to about 1:1.5. In other embodiments the weight ratio of total amounts of color-removing agent(s): keto acid(s) may be about 0.1, about 0.2, about 0.3, about 0.4, about 0.5, about 0.6, about 0.7, about 0.8, about 0.9, about 1, about 1.1, about 1.2, about 1.3, about 1.4, or about 1.5, including all ranges and subranges using any of the foregoing as upper and lower limits

In embodiments where the hair color-removing agents are chosen from thiol-based compounds, e.g. thiosalicylic acid, thiolactic acid, and/or salts thereof, the weight ratio of total amounts of thiol-based color-removing agent(s): keto acid(s) can be any of the aforementioned ratios. For example, in some embodiments the weight ratio of thiol-based color-removing agent(s): keto acid(s) can range from about 1:0.5 to 1:10, or can be equal to or less than 1. For example, the weight ratio of total amounts of thiol-based color-removing agent(s): keto acid(s) may range from about 1:1 to about 1:10, such as from about 1:1 to about 1:8, from about 1:1 to about 1:6, from about 1:1 to about 1:5, from about 1:1 to about 1:4, from about 1:1 to about 1:3, from about 1:1 to about 1:2, from about 1:1 to about 1:1.5, from about 1:1.1 to about 1:10, about 1:1.1 to about 1:8, from about 1:1.1 to about 1:6, from about 1:1.1 to about 1:5, from about 1:1.1 to about 1:4, from about 1:1.1 to about 1:3, from about 1:1.1 to about 1:2, or from about 1:1.1 to about 1:1.5. In other embodiments the weight ratio of total amounts of thiol-based color-removing agent(s): keto acid(s) may be about 0.1, about 0.2, about 0.3, about 0.4, about 0.5, about 0.6, about 0.7, about 0.8, about 0.9, about 1, about 1.1, about 1.2, about 1.3, about 1.4, or about 1.5, including all ranges and subranges using any of the foregoing as upper and lower limits.

### Surfactants

Optionally, hair-color removing compositions that can be used may comprise at least one surfactant, which may in various embodiments be chosen from anionic, non-ionic, amphoteric, and/or cationic surfactants. For example, the hair-color removing compositions may comprise one or more anionic surfactants, one or more non-ionic surfactants, or one or more amphoteric surfactants, one or more cationic surfactants, or the compositions may comprise mixtures of surfactants having the same or different ionicities. Salts of surfactants are included whether or not expressly stated, unless specified otherwise.

### Anionic Surfactants

In at least some embodiments, the hair-color removing compositions comprise at least at least one anionic surfactant. The term “anionic surfactant” means a surfactant comprising, as ionic or ionizable groups, only anionic groups. A species is termed as being “anionic” when it bears at least one permanent negative charge or when it can be ionized as a negatively charged species, under the conditions of use of the composition (for example the medium or the pH) and not comprising any cationic charge. These anionic groups may be chosen from, for example, —CO₂H, —CO₂⁻, —SO₃H, —SO₃⁻, —OSO₃H, —OSO₃⁻, —H₂PO₃, —HPO₃⁻, —PO₃²⁻, —H₂PO₂, ═HPO₂, —HPO₂⁻, ═PO₂⁻, ═POH, and ═PO⁻ groups.

The anionic surfactants may be sulfate, sulfonate, and/or carboxylic (or carboxylate) surfactants, or mixtures thereof.

Sulfate anionic surfactants comprise at least one sulfate function (—OSO₃H or —OSO₃) but generally do not comprise any carboxylate or sulfonate functions. In some embodiments the sulfate anionic surfactants that are used are free of carboxylate or sulfonate functions.

Non-limiting examples of sulfate anionic surfactants include alkyl sulfates, alkyl ether sulfates, alkylamido ether sulfates, alkylaryl polyether sulfates, monoglyceride sulfates; and also the salts of these compounds; the alkyl groups of these compounds comprising from 6 to 30 carbon atoms, especially from 12 to 28, better still from 14 to 24 or even from 16 to 22 carbon atoms; the aryl group preferably denoting a phenyl or benzyl group; these compounds possibly being polyoxyalkylenated, for example polyoxyethylenated, and optionally comprising from 1 to 50 ethylene oxide units such as from 2 to 10 ethylene oxide units.

Sulfonate anionic surfactants comprise at least one sulfonate function (—SO₃H or —SO₃⁻) and may optionally also comprise one or more sulfate functions, but preferably do not comprise any carboxylate functions.

Non-limiting examples of sulfonate anionic surfactants include alkylsulfonates, alkylamidesulfonates, alkylarylsulfonates, α-olefinsulfonates, paraffin sulfonates, alkylsulfosuccinates, alkyl ether sulfosuccinates, alkylamidesulfosuccinates, alkylsulfoacetates, N-acyltaurates, acylisethionates; alkylsulfolaurates; and also the salts of these compounds; the alkyl groups of these compounds comprising from 6 to 30 carbon atoms, for example from 12 to 28, such as from 14 to 24 or even from 16 to 22 carbon atoms; the aryl group preferably denoting a phenyl or benzyl group; these compounds possibly being polyoxyalkylenated, preferably polyoxyethylenated, and optionally comprising from 1 to 50 ethylene oxide units and better still from 2 to 10 ethylene oxide units.

Carboxylate anionic surfactants comprise at least one carboxylic or carboxylate function (—COOH or —COO⁻) and may optionally also comprise one or more sulfate and/or sulfonate functions.

Non-limiting examples of carboxylate anionic surfactants include acylglycinates, acyllactylates, acylsarcosinates, acylglutamates, alkyl-D-galactosideuronic acids, alkyl ether carboxylic acids, alkyl(C6-30 aryl) ether carboxylic acids, alkylamido ether carboxylic acids; and also the salts of these compounds; the alkyl and/or acyl groups of these compounds comprising from 6 to 30 carbon atoms, for example from 12 to 28, such as from 14 to 24 or even from 16 to 22 carbon atoms; the aryl group preferably denoting a phenyl or benzyl group; these compounds possibly being polyoxyalkylenated, preferably polyoxyethylenated, and optionally comprising from 1 to 50 ethylene oxide units and better still from 2 to 10 ethylene oxide units.

When the anionic surfactant is in salt form, the salt may be chosen from alkali metal salts, such as the sodium or potassium salt, ammonium salts, amine salts and in particular amino alcohol salts, and alkaline-earth metal salts, such as the magnesium salt.

Examples of amino alcohol salts may include but are not limited to monoethanolamine, diethanolamine, and triethanolamine salts, monoisopropanolamine, diisopropanolamine, and triisopropanolamine salts, 2-amino-2-methyl-1-propanol salts, 2-amino-2-methyl-1,3-propanediol salts, and tris(hydroxymethyl) aminomethane salts.

In some embodiments, alkali metal or alkaline-earth metal salts and in particular the sodium or magnesium salts may be chosen, for example in the form of alkali metal, ammonium, amino alcohol and alkaline-earth metal salts, or a mixture of these compounds, may be chosen.

In exemplary and non-limiting embodiments, the anionic surfactant may be chosen from sodium laureth sulfate, ammonium laureth sulfate, disodium lauryl sulfosuccinate, disodium laureth sulfosuccinate, diammonium lauryl sulfosuccinate, diethylhexyl sodium sulfosuccinate, sodium oleyl succinate, sodium lauroyl methyl isethionate, sodium lauryl isethionate, sodium cocoyl isethionate, sodium laureth-5 carboxylate, lauryl ether carboxylic acid, ammonium lauryl sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, potassium lauryl sulfate, potassium laureth sulfate, ammonium cocoyl sulfate, ammonium lauroyl sulfate, sodium cocoyl sulfate, sodium lauroyl sulfate, potassium cocoyl sulfate, monoethanolamine cocoyl sulfate, sodium tridecyl benzene sulfonate, sodium dodecyl benzene sulfonate, sodium C₁₄₋₁₆ Olefin sulfonate, sodium lauryl sarcosinate, sodium lauroyl sarcosinate, stearoyl sarcosine, lauryl sarcosine, cocoyl sarcosine, sodium methyl cocoyl taurate, sodium methyl lauroyl taurate, sodium lauroyl glutamate, sodium cocoyl glutamate, disodium cocoyl glutamate, potassium myristoyl glutamate, TEA-cocoyl glutamate, sodium cocoyl glycinate, potassium cocoyl glycinate, sodium cocoyl alaninate, TEA-cocoyl alaninate, or a combination of two or more thereof. For example, the compositions may comprise at least one anionic surfactant chosen from sodium laureth sulfate, sodium lauryl sulfate, sodium lauroyl sulfate, sodium lauroyl methyl isethionate, or a combination of two or more thereof.

If present, the total amount of anionic surfactants may range from about 0.01% up to about 15%, such as up to about 12%, up to about 10%, up to about 8%, up to about 5%, up to about 3.5%, or up to about 2% by weight, relative to the total weight of the composition. For example, the total amount of anionic surfactants may range from about 0.01% to about 12%, from about 0.1% to about 10%, from about 0.5% to about 8%, or from about 1% to about 6% by weight, relative to the total weight of the hair-color removing composition. In at least some embodiments, the compositions comprise at least one anionic surfactant, and have a total amount of anionic surfactants ranging from about 0.5% to about 10%, from about 0.5% to about 8%, from about 0.5% to about 6%, from about 0.5% to about 5%, from about 0.5% to about 4%, from about 0.5% to about 3%, from about 0.75% to about 10%, from about 0.75% to about 8%, from about 0.75% to about 6%, from about 0.75% to about 5%, from about 0.75% to about 4%, from about 0.75% to about 3%, from about 1% to about 10%, from about 1% to about 8%, from about 1% to about 6%, from about 1% to about 5%, from about 1% to about 4%, from about 1% to about 3%, from about 1.25% to about 10%, from about 1.25% to about 8%, from about 1.25% to about 6%, from about 1.25% to about 5%, from about 1.25% to about 4%, from about 1.25% to about 3%, from about 1.5% to about 10%, from about 1.5% to about 8%, from about 1.5% to about 6%, from about 1.5% to about 5%, from about 1.5% to about 4%, or from about 1.5% to about 3%, by weight relative to the total weight of the composition. In various embodiments, the total amount of anionic surfactant may be about 0.25%, about 0.5%, about 0.75%, about 1%, about 1.25%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight, relative to the total weight of the composition, or may be present in any range using any of the foregoing as upper and lower limits.

### Amphoteric Surfactants

In at least some embodiments, the hair-color removing compositions comprise at least one amphoteric (also referred to as zwitterionic surfactants), although in at least some embodiments the compositions are free or essentially free of amphoteric surfactants. Non-limiting examples of useful amphoteric surfactants include derivatives of aliphatic secondary and tertiary amines where the aliphatic radical can be straight or branched chain and one of the aliphatic substituents contains from about 8 to about 18 carbon atoms and one contains an anionic group such as carboxy, sulfonate, sulfate, phosphate, or phosphonate. Exemplary amphoteric surfactants include sodium cocaminopropionate, sodium cocaminodipropionate, sodium cocoamphoacetate, sodium cocoamphohydroxypropylsulfonate, sodium cocoamphopropionate, sodium cornamphopropionate, sodium lauraminopropionate, sodium lauroamphoacetate, sodium lauroamphohydroxypropylsulfonate, sodium lauroamphopropionate, sodium cornamphopropionate, sodium lauriminodipropionate, ammonium cocaminopropionate, ammonium cocaminodipropionate, ammonium cocoamphoacetate, ammonium cocoamphohydroxypropylsulfonate, ammonium cocoamphopropionate, ammonium cornamphopropionate, ammonium lauraminopropionate, ammonium lauroamphoacetate, ammonium lauroamphohydroxypropylsulfonate, ammonium lauroamphopropionate, ammonium cornamphopropionate, ammonium lauriminodipropionate, triethanonlamine cocaminopropionate, triethanonlamine cocaminodipropionate, triethanonlamine cocoamphoacetate, triethanonlamine cocoamphohydroxypropylsulfonate, triethanonlamine cocoamphopropionate, triethanonlamine cornamphopropionate, triethanonlamine lauraminopropionate, triethanonlamine lauroamphoacetate, triethanonlamine lauroamphohydroxypropylsulfonate, triethanonlamine lauroamphopropionate, triethanonlamine cornamphopropionate, triethanonlamine lauriminodipropionate, cocoamphodipropionic acid, disodium caproamphodiacetate, disodium caproamphoadipropionate, disodium capryloamphodiacetate, disodium capryloamphodipriopionate, disodium cocoamphocarboxyethylhydroxypropylsulfonate, disodium cocoamphodiacetate, disodium cocoamphodipropionate, disodium dicarboxyethylcocopropylenediamine, disodium laureth-5 carboxyamphodiacetate, disodium lauriminodipropionate, disodium lauroamphodiacetate, disodium lauroamphodipropionate, disodium oleoamphodipropionate, disodium PPG-2-isodecethyl-7 carboxyamphodiacetate, lauraminopropionic acid, lauroamphodipropionic acid, lauryl aminopropylglycine, and lauryl diethylenediaminoglycine, as well as combinations of two or more thereof.

Betaines may also be used. For example, coco dimethyl carboxymethyl betaine, lauryl dimethyl carboxymethyl betaine, lauryl dimethyl alphacarboxyethyl betaine, cetyl dimethyl carboxymethyl betaine, cetyl dimethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl) alpha-carboxyethyl betaine, coco dimethyl sulfopropyl betaine, stearyl dimethyl sulfopropyl betaine, lauryl dimethyl sulfoethyl betaine, lauryl bis-(2-hydroxyethyl) sulfopropyl betaine, oleyl betaine, cocamidopropyl betaine, or combinations of two or more thereof, may be chosen.

If present, the total amount of amphoteric surfactants may range from about 0.01% up to about 15%, such as up to about 12%, up to about 10%, up to about 8%, up to about 5%, up to about 3.5%, or up to about 2% by weight, relative to the total weight of the composition. For example, the total amount of amphoteric surfactants may range from about 0.01% to about 10%, from about 0.1% to about 8%, from about 0.5% to about 6%, or from about 1% to about 4% by weight, relative to the total weight of the composition. In at least some embodiments, the compositions comprise at least one amphoteric surfactant, and have a total amount of amphoteric or zwitterionic surfactants ranging from about 0.25% to about 5%, such as from about 0.5% to about 4%, from about 0.75% to about 3%, or from about 1% to about 2%, or may be about 0.25%, about 0.5%, about 0.75%, about 1%, about 1.25%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight, relative to the total weight of the hair-color removing composition, including any range using any of the foregoing as upper and lower limits. In at least some other embodiments, the compositions are free or substantially free of amphoteric surfactants.

### Nonionic Surfactants

In at least some embodiments, the compositions comprise at least one nonionic surfactant, although in at least some embodiments the compositions are free or essentially free of nonionic surfactants. The nonionic surfactants may be chosen from alcohols, α-diols and (C₁-C₂₀) alkylphenols, these compounds being polyethoxylated, polypropoxylated and/or polyglycerolated, the number of ethylene oxide and/or propylene oxide groups possibly ranging from 1 to 100, and the number of glycerol groups possibly ranging from 2 to 30, or alternatively these compounds comprising at least one fatty chain comprising from 8 to 30 carbon atoms and especially from 16 to 30 carbon atoms. For example, nonionic surfactants may be chosen from monooxyalkylenated or polyoxyalkylenated (C₈-C₂₄) alkylphenols, saturated or unsaturated, linear or branched, monooxyalkylenated or polyoxyalkylenated C₈-C₃₀ alcohols, saturated or unsaturated, linear or branched, monooxyalkylenated or polyoxyalkylenated C₈-C₃₀ amides, esters of saturated or unsaturated, linear or branched, C₈-C₃₀ acids and of polyalkylene glycols, monooxyalkylenated or polyoxyalkylenated esters of saturated or unsaturated, linear or branched, C₈-C₃₀ acids and of sorbitol, saturated or unsaturated, monooxyalkylenated or polyoxyalkylenated plant oils, condensates of ethylene oxide and/or of propylene oxide, or combinations thereof.

By way of example only, the adducts of ethylene oxide with lauryl alcohol, for example those containing from 9 to 50 oxyethylene units or from 10 to 12 oxyethylene units (Laureth-10 to Laureth-12); the adducts of ethylene oxide with behenyl alcohol, for example those containing from 9 to 50 oxyethylene units (Beheneth-9 to Beheneth-50); the adducts of ethylene oxide with cetearyl alcohol (mixture of cetyl alcohol and stearyl alcohol), for example those containing from 10 to 30 oxyethylene units (Ceteareth-10 to Ceteareth-30); the adducts of ethylene oxide with cetyl alcohol, for example those containing from 10 to 30 oxyethylene units (Ceteth-10 to Ceteth-30); the adducts of ethylene oxide with stearyl alcohol, for example those containing from 10 to 30 oxyethylene units (Steareth-10 to Steareth-30); the adducts of ethylene oxide with isostearyl alcohol, for example those containing from 10 to 50 oxyethylene units (Isosteareth-10 to Isosteareth-50); monoglycerolated or polyglycerolated C₈-C₄₀, e.g. C₈-C₃₀, alcohols, such as lauryl alcohol containing 4 mol of glycerol (Polyglyceryl-4 Lauryl Ether), lauryl alcohol containing 1.5 mol of glycerol, oleyl alcohol containing 4 mol of glycerol (Polyglyceryl-4 Oleyl Ether), oleyl alcohol containing 2 mol of glycerol (Polyglyceryl-2 Oleyl Ether), cetearyl alcohol containing 2 mol of glycerol, cetearyl alcohol containing 6 mol of glycerol, oleocetyl alcohol containing 6 mol of glycerol, or octadecanol containing 6 mol of glycerol; polyoxyethylenated fatty esters such as the adducts of ethylene oxide with esters of lauric acid, palmitic acid, stearic acid or behenic acid, and mixtures thereof, for example those containing from 9 to 100 oxyethylene units such as PEG-9 to PEG-50 laurate, PEG-9 to PEG-50 palmitate, PEG-9 to PEG-50 stearate, PEG-9 to PEG-50 palmitostearate, PEG-9 to PEG-50 behenate, polyethylene glycol 100 EO monostearate (PEG-100 stearate); glyceryl stearate (glyceryl mono-, di- and/or tristearate); glyceryl ricinoleate; sorbitan palmitate; sorbitan isostearate; sorbitan stearate; sorbitan palmitate; sorbitan trioleate; alkylglucose sesquistearates such as methylglucose sesquistearatel alkylglucose palmitates such as methylglucose or ethylglucose palmitate, etc., or combinations of two or more thereof may be chosen.

If present, the total amount of nonionic surfactants may range from about 0.01% up to about 15%, such as up to about 12%, up to about 10%, up to about 8%, up to about 5%, up to about 3.5%, or up to about 2% by weight, relative to the total weight of the hair-color removing composition. For example, the total amount of nonionic surfactants may range from about 0.01% to about 10%, from about 0.1% to about 8%, from about 0.5% to about 6%, or from about 1% to about 4% by weight, relative to the total weight of the composition. In at least some embodiments, the compositions comprise at least one nonionic surfactant, and have a total amount of nonionic surfactants ranging from about 0.25% to about 5%, such as from about 0.5% to about 4%, from about 0.75% to about 3%, or from about 1% to about 2%, or may be about 0.25%, about 0.5%, about 0.75%, about 1%, about 1.25%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight, relative to the total weight of the composition, including any range using any of the foregoing as upper and lower limits. In at least some embodiments, the compositions are free or substantially free of nonionic surfactants.

### Cationic Surfactants

In at least some embodiments, the hair-color removing compositions comprise at least one cationic surfactant, although in at least some embodiments the compositions are free or essentially free of cationic surfactants. Exemplary and non-limiting cationic surfactants include cationic amine-based or quaternary ammonium-based compounds.

For example, cationic surfactants may be chosen from alkylpyridinium salts, ammonium salts of imidazoline, diquaternary ammonium salts, and ammonium salts containing at least one ester function.

The surfactants may be, for example, the salts (chloride or methyl sulfate) of diacyloxyethyldimethylammonium, of diacyloxyethylhydroxyethyldimethylammonium, of monoacyloxyethylhydroxyethyldimethylammonium, of triacyloxyethylmethylammonium, of monoacyloxyethylhydroxyethyldimethylammonium, and mixtures thereof. The acyl radicals preferably contain 14 to 18 carbon atoms and are more particularly derived from a plant oil, for instance palm oil or sunflower oil. When the compound contains several acyl radicals, these radicals may be identical or different.

Other suitable cationic surfactants are esterquats which are quaternary ammonium compounds having fatty acid chains containing ester linkages, such as, for example, dibehenoylethyl dimonium chloride, dipalmitoylethyl dimonium chloride, distearoylethyl dimonium chloride, ditallowoyl PG-dimonium chloride, dipalmitoylethyl hydroxyethylmonium methosulfate, distearoylethyl hydroxyethylmonium methosulfate, or mixtures thereof.

Additional non-limiting examples of useful cationic surfactants include brassicamidopropyl dimethylamine, behentrimonium chloride, cetrimonium chloride, behenalkonium chloride, benzethonium chloride, cetylpyridinium chloride, behentrimonium chloride, lauralkonium chloride, cetalkonium chloride, cetrimonium bromide, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3) oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, hexadecyltrimethyl ammonium bromide, stearamidopropyl dimethylamine, or combinations thereof.

If present, the total amount of cationic surfactants may range from about 0.01% up to about 15%, such as up to about 12%, up to about 10%, up to about 8%, up to about 5%, up to about 3.5%, or up to about 2% by weight, relative to the total weight of the hair-color removing composition. For example, the total amount of cationic surfactants may range from about 0.01% to about 10%, from about 0.1% to about 8%, from about 0.5% to about 6%, or from about 1% to about 4% by weight, relative to the total weight of the composition. In at least some embodiments, the compositions comprise at least one cationic surfactant, and have a total amount of cationic surfactants ranging from about 0.25% to about 5%, such as from about 0.5% to about 4%, from about 0.75% to about 3%, or from about 1% to about 2%, or may be about 0.25%, about 0.5%, about 0.75%, about 1%, about 1.25%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight, relative to the total weight of the composition, including any range using any of the foregoing as upper and lower limits. In some embodiments, however, the compositions are free or essentially free of cationic surfactants.

### Clay Compounds

Hair color removing compositions that can be used in methods according to the disclosure optionally comprise at least one clay compound, although in at least some embodiments the hair-color removing compositions are free or essentially free of clay compounds. In some embodiments, the compositions comprise more than one clay compound, for example, at least two clay compounds, etc. Without intending to be limiting, it is believed that addition of one or more clay compounds may aid in preventing re-oxidation of the dyes, thus making the color-removal long lasting.

By way of non-limiting example, the clay compound may be chosen from kaolinite (also referred to interchangeably as kaolin), bentonite, magnesium aluminum silicate, hectorite, smectite, vermiculite, illite, chorite, halloysite, palygorskite, sepiolite, sesquioxide, imogolite, allophane, or combinations of two or more thereof. For example, in one embodiment, kaolin is chosen. In another embodiment, bentonite is chosen. In various embodiments, the clay compound comprises, consists essentially of, or consists of smectite, hectorite, kaolin, bentonite, magnesium aluminum silicate, or a combination of two or more thereof.

If present, the total amount of clay can range from about 0.1% to about 15%, such as from about 0.5% to about 12%, from about 1% to about 10%, or from about 2% to about 8% by weight, relative to the total weight of the composition. For example, the total amount of clay may range from about 1% to about 12%, from about 1% to about 11%, from about 1% to about 10%, from about 1% to about 9%, from about 1% to about 8%, from about 1% to about 7%, from about 1% to about 6%, from about 1% to about 5%, from about 1% to about 4%, from about 1% to about 3%, from about 1% to about 2%, from about 2% to about 12%, from about 2% to about 11%, from about 2% to about 10%, from about 2% to about 9%, from about 2% to about 8%, from about 2% to about 7%, from about 2% to about 6%, from about 2% to about 5%, from about 2% to about 4%, from about 2% to about 3%, from about 3% to about 12%, from about 3% to about 11%, from about 3% to about 10%, from about 3% to about 9%, from about 3% to about 8%, from about 3% to about 7%, from about 3% to about 6%, from about 3% to about 5%, from about 3% to about 4%, from about 4% to about 12%, from about 4% to about 11%, from about 4% to about 10%, from about 4% to about 9%, from about 4% to about 8%, from about 4% to about 7%, from about 4% to about 6%, from about 4% to about 5%, from about 5% to about 12%, from about 5% to about 11%, from about 5% to about 10%, from about 5% to about 9%, from about 5% to about 8%, from about 5% to about 7%, from about 5% to about 6%, from about 6% to about 12%, from about 6% to about 11%, from about 6% to about 10%, from about 6% to about 9%, from about 6% to about 8%, from about 6% to about 7%, from about 7% to about 12%, from about 7% to about 11%, from about 7% to about 10%, from about 7% to about 9%, from about 7% to about 8%, from about 8% to about 12%, from about 8% to about 11%, from about 8% to about 10%, from about 8% to about 9%, from about 9% to about 12%, from about 9% to about 11%, from about 9% to about 10%, from about 10% to about 12%, or from about 10% to about 11% by weight, relative to the total weight of the hair-color removing composition. For example, the total amount of clays may be about 1%, about 2%, about 3%, about 4%, about 5%, about 6%, about 7%, about 8%, about 9%, about 10%, about 11%, or about 12% by weight, relative to the total weight of the composition, including any range using any of the foregoing as upper and lower limits.

As a non-limiting example, the clay compound may comprise, consist essentially of, or consist of bentonite, kaolin, smectite, hectorite, or a combination of two or more thereof, and the total amount of clays may be about 1%, about 2%, about 3%, about 4%, about 5%, about 6%, about 7%, about 8%, about 9%, about 10%, about 11%, or about 12% by weight, relative to the total weight of the composition, or may be present in a range using any of the foregoing as upper or lower limits.

### Fatty Compounds

Optionally, hair-color removing compositions that can be used may include at least one fatty compound, although in at least some embodiments the compositions are free or essentially free of fatty compounds. In certain embodiments, the at least one fatty compound may be chosen from lower alkanes, fatty alcohols, fatty acids, esters of fatty acids, esters of fatty alcohols, oils such as mineral, vegetable, animal, silicone and non-silicone oils, silicone and non-silicone waxes, or combinations of any two or more thereof. In some embodiments, the hair-color removing compositions comprise at least one fatty compound of natural origin. In some embodiments, the hair-color removing compositions are free or essentially free of fatty compounds that are not of natural origin. In some embodiments, the compositions are free or essentially free of silicone fatty compounds.

If present, silicone oils may, for example, be chosen from polydimethylsiloxanes (PDMSs), polydimethylsiloxanes comprising alkyl or alkoxy groups which are pendent and/or at the end of the silicone chain, which groups each contain from 2 to 24 carbon atoms, or phenyl silicones, such as phenyl trimethicones, phenyl dimethicones, phenyl(trimethylsiloxy) diphenylsiloxanes, diphenyl dimethicones, diphenyl(methyl-diphenyl)trisiloxanes, or (2-phenylethyl)trimethyl-siloxysilicates. Non-limiting examples of silicone oils include dimethicone, amodimethicone, cyclomethicone, polysilicone-11, phenyl trimethicone, trimethylsilylamodimethicone, and stearoxytrimethylsilane. For example, the composition may comprise at least one silicone chosen from amodimethicone, PEG-7 Dimethicone, PEG-8 Dimethicone, PEG-9 Dimethicone, PEG-10 Dimethicone, PEG-12 Dimethicone, PEG-14 Dimethicone, PEG-17 Dimethicone, PEG/PPG-3/10 Dimethicone, PEG/PPG-4/12 Dimethicone, PEG/PPG-17/18 Dimethicone, cetyl PEG/PPG-10/1 dimethicone, Dimethicone PEG-8 Benzoate, Dimethicone PEG-7 Phosphate, Dimethicone PEG-8 Phosphate, Dimethicone PEG-10 Phosphate, or a combination of two or more thereof.

In some embodiments, the hair-color removing compositions comprise a silicone oil component that comprises, consists essentially of, or consists of dimethicone, amodimethicone, or a combination thereof. In some embodiments, the hair-color removing compositions are free or substantially free of silicone oils and/or silicone waxes.

In some embodiments, hair-color removing compositions according to the disclosure may include at least one fatty compound chosen from fatty alcohols. Herein, “fatty alcohol” can refer to any alcohol with a carbon chain of C5 or greater, such as, for example, C8 or greater, C10 or greater, or C12 or greater, such as from 6 to 30 carbon atoms or from 8 to 30 carbon atoms. The fatty alcohols may be alkoxylated or non-alkoxylated, saturated or unsaturated, and linear or branched. Non-limiting examples of fatty alcohols include arachidyl alcohol, behenyl alcohol, caprylic alcohol, cetearyl alcohol, cetyl alcohol, coconut alcohol, decyl alcohol, hydrogenated tallow alcohol, jojoba alcohol, lauryl alcohol, myristyl alcohol, oleyl alcohol, palm alcohol, palm kernel alcohol, stearyl alcohol, tallow alcohol, tridecyl alcohol, or combinations of two or more thereof. In some preferred embodiments, the compositions comprise a fatty alcohol component that comprises, consists essentially of, or consists of cetyl alcohol, stearyl alcohol, cetearyl alcohol, or combinations thereof.

Useful and non-limiting fatty acids may be straight or branched chain acids and/or may be saturated or unsaturated. Non-limiting examples of fatty acids include diacids, triacids, and other multiple acids as well as salts of these fatty acids. For example, the fatty acid may optionally include or be chosen from lauric acid, palmitic acid, stearic acid, behenic acid, arichidonic acid, oleic acid, isostearic acid, sebacic acid, or combinations thereof.

In some embodiments, fatty acid esters or fatty alcohol esters may be chosen. For example, esters of saturated or unsaturated, linear or branched C1-C26 aliphatic mono- or polyacids and of saturated or unsaturated, linear or branched C1-C26 aliphatic mono- or polyalcohols, the total carbon number of the esters more particularly being greater than or equal to 10 may be used. In other embodiments, esters of C4-C22 dicarboxylic or tricarboxylic acids and of C1-C22 alcohols and esters of mono-, di- or tricarboxylic acids and of C2-C26 di-, tri-, tetra-, or pentahydroxy alcohols may also be used. As non-limiting examples, isostearyl lactate, lauryl lactate, linoleyl lactate, oleyl lactate, (iso) stearyl octanoate, isocetyl octanoate, octyl octanoate, cetyl octanoate, decyl oleate, isocetyl isostearate, isocetyl laurate, isocetyl stearate, isodecyl octanoate, isodecyl oleate, isononyl isononanoate, isostearyl palmitate, methyl acetyl ricinoleate, myristyl stearate, octyl isononanoate, 2-ethylhexyl isononanoate, octyl palmitate, octyl pelargonate, octyl stearate, octyldodecyl erucate, oleyl erucate, ethyl and isopropyl palmitates, 2-ethylhexyl palmitate, 2-octyldecyl palmitate, alkyl myristates such as isopropyl, butyl, cetyl, 2-octyldodecyl, myristyl or stearyl myristate, hexyl stearate, butyl stearate, isobutyl stearate, dioctyl malate, hexyl laurate, 2-hexyldecyl laurate, diethyl sebacate, diisopropyl sebacate, diisopropyl adipate, di-n-propyl adipate, dioctyl adipate, diisostearyl adipate, dioctyl maleate, glyceryl undecylenate, octyldodecyl stearoyl stearate, pentaerythrityl monoricinoleate, pentaerythrityl tetraisononanoate, pentaerythrityl tetrapelargonate, pentaerythrityl tetraisostearate, pentaerythrityl tetraoctanoate, propylene glycol dicaprylate, propylene glycol dicaprate, tridecyl erucate, triisopropyl citrate, triisostearyl citrate, glyceryl trilactate, glyceryl trioctanoate, trioctyldodecyl citrate, trioleyl citrate, propylene glycol dioctanoate, neopentyl glycol diheptanoate, diethylene glycol diisononanoate, and/or polyethylene glycol distearates may be chosen. In a preferred embodiment, the composition comprises at least one fatty acid ester and/or fatty alcohol ester, for example pentaerythrityl tetraisostearate.

Non-limiting examples of waxes that can be used include beeswax, hydrogenated alkyl olive esters, carnauba wax, candelilla wax, ouricoury wax, Japan wax, cork fibre wax or sugar cane wax, rice wax, rice bran wax, montan wax, paraffin wax, lignite wax or microcrystalline wax, ceresin or ozokerite, palm kernel glycerides/hydrogenated palm glycerides, palm butter, sumac wax, citrus aurantium dulcis (orange) peel wax, theobroma grandiflorum seed butter, Helianthus annuus (sunflower) seed wax, siliconyl candellila wax, Chinese wax, cetyl palmitate, lanolin, shellac, spermaceti, cetyl esters, hydrogenated castor wax; triglyceride esters such as tribehenin (glyceryl tribehenate); synthetic waxes such as those of the hydrocarbon type and polyethylene waxes obtained from the polymerization or copolymerization of ethylene, polypropylene waxes, or combinations of two or more of any of these waxes. However, in some embodiments the compositions are free or essentially free of waxes.

In some embodiments, the hair-color removing composition may include one or more fatty compounds chosen from oils of animal, vegetable, or mineral origin (e.g. lanolin, squalene, fish oil, perhydrosqualene, mink oil, turtle oil, soybean oil, grape seed oil, sesame oil, maize oil, rapeseed oil, sunflower oil, cottonseed oil, avocado oil, olive oil, castor seed oil, jojoba seed oil, peanut oil, sweet almond oil, palm oil, cucumber oil, hazelnut oil, apricot kernel oil, wheat germ oil, calophyllum oil, macadamia oil, coconut oil, cereal germ oil, candlenut oil, thistle oil, candelilla oil, safflower oil, or shea butter), linear or branched hydrocarbons (e.g. polybutene, hydrogenated polyisobutene, polyisoprene, polydecenes such as hydrogenated polydecene, or also linear, branched and/or cyclic alkanes which are optionally volatile, such as, for example, isohexadecane, isododecane, isodecane, or isohexadecane), mono- and/or polyesters of fatty acids and/or of fatty alcohols (e.g. mono- and polyesters of hydroxy acids and of fatty alcohols, esters of benzoic acid and of fatty alcohols, polyesters of polyols, dipentaerythrityl C5-C9 esters, trimethylolpropane polyesters, propylene glycol polyesters, or polyesters of hydrogenated castor oil), perfluorinated and/or organofluorinated oils, fluorosilicone oils, or combinations of two or more thereof. In some embodiments, the compositions comprise at least one oil, e.g. mineral oil.

If present, the total amount of fatty compounds in the hair-color removing composition may range from about 0.01% up to about 15%, such as up to about 12%, up to about 10%, up to about 9%, up to about 8%, up to about 7%, up to about 6%, up to about 5%, or up to about 4% by weight, relative to the total weight of the composition. For example, the total amount of fatty compounds may range from about 0.1% to about 12%, from about 0.25% to about 10%, from about 0.5% to about 8%, from about 0.75% to about 6%, or from about 1% to about 5% by weight, relative to the total weight of the composition. In at least some embodiments, however, the compositions are free or essentially free of fatty compounds.

### Thickening Agents

Hair color-removing compositions that can be used in methods according to the disclosure optionally comprise at least one thickening agent. Useful thickening agents include, but are not limited to, semisynthetic polymers, such as semisynthetic cellulose derivatives, synthetic polymers, such as carbomers, poloxamers, and acrylates/beheneth-25 methacrylate copolymer, acrylates copolymer, polyethyleneimines (e.g., PEI-10), naturally occurring polymers, such as acacia, tragacanth, alginates (e.g., sodium alginate), carrageenan, vegetable gums, such as xanthan gum, guar gum, petroleum jelly, waxes, particulate associate colloids, such as bentonite, colloidal silicon dioxide, and microcrystalline cellulose, celluloses such as hydroxyethylcellulose and hydroxypropylcellulose, and guars such as hydroxypropyl guar.

In some embodiments, the thickening agent may be chosen from associative thickening polymers such as anionic associative polymers, amphoteric associative polymers, cationic associative polymers, or nonionic associative polymers. A non-limiting example of an amphoteric associative polymer is acrylates/beheneth-25 methacrylate copolymer, and non-limiting examples of anionic associative polymers include acrylates copolymer and acrylates crosspolymer-4.

If present, the total amount of thickening agents may range from about 0.001% to about 5%, such as from about 0.01% to about 4%, from about 0.1% to about 3.5%, from about 0.2% to about 3%, from about 0.3% to about 2.5%, from about 0.4% to about 2%, from about 0.5% to about 1.5%, or from about 0.5% to about 1% by weight, relative to the total weight of the hair-color removing composition.

### Amino Acids and Amino Sulfonic Acids

The hair-color removing compositions that can be used may optionally comprise at least one amino acid and/or amino sulfonic acid, although in at least some embodiments the compositions are free or essentially free of amino acids and/or amino sulfonic acids. Salts of amino acids and amino sulfonic acids may also be chosen, and are expressly included whether or not so stated.

Amino acids that may be chosen include, for example, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. Exemplary amino sulfonic acids include aminomethane sulfonic acid, 2-aminoethane sulfonic acid (taurine), aminopropane sulfonic acid, aminobutane sulfonic acid, aminohexane sulfonic acid, aminoisopropyl sulfonic acid, aminododecyl sulfonic acid, aminobenzene sulfonic acid, aminotoulene sulfonic acid, sulfanilic acid, chlorosulfanilic acid, diamino benzene sulfonic acid, amino phenol sulfonic acid, amino propyl benzene sulfonic acid, and amino hexyl benzene sulfonic acid. In some embodiments, the hair treatment compositions comprise at least one amino acid and at least one amino sulfonic acid. In some embodiments, the composition comprises arginine, serine, betaine, and/or taurine, for example arginine, betaine, or a combination thereof. For example, in some embodiments, arginine, serine, betaine, and/or taurine are the only amino acids and amino sulfonic acids present in the compositions, and in some embodiments, arginine and/or betaine are the only amino acids and amino sulfonic acids present in the compositions.

If present, the total amount of amino acids and/or amino sulfonic acids may range from about 0.01% to about 10%, such as from about 0.1% to about 8%, or from about 0.5% to about 6% by weight, relative to the total weight of the hair-color removing composition. For example, in some embodiments, the total amount of amino acid(s) and/or amino sulfonic acid(s) may range from about 0.5% to about 5%, from about 0.5% to about 4%, from about 0.5% to about 3%, from about 0.5% to about 2.5%, from about 0.5% to about 2%, from about 0.5% to about 1.5%, from about 0.5% to about 1%, from about 0.75% to about 5%, from about 0.75% to about 4%, from about 0.75% to about 3%, from about 0.75% to about 2.5%, from about 0.75% to about 2%, from about 0.75% to about 1.5%, from about 0.75% to about 1%, from about 1% to about 5%, from about 1% to about 4%, from about 1% to about 3%, from about 1% to about 2.5%, from about 1% to about 2%, or from about 1% to about 1.5% by weight, relative to the total weight of the composition. In some embodiments, the total amount of amino acid(s) and/or amino sulfonic acid(s) may be about 0.25%, about 0.5%, about 0.75%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, or about 10% by weight, relative to the total weight of the composition, including all ranges and subranges using any of the foregoing as upper and lower limits.

### Organic Amine Compounds

In some embodiments, the hair-color removing compositions optionally comprise at least one organic amine compound, although in at least some embodiments the compositions are free or essentially free of organic amine compounds. Optionally, the hair-color removing compositions may comprise more than one organic amine compound, such as at least two organic amine compounds.

In various embodiments, alkanolamines, such as mono-, di-, or trialkanolamines, comprising from one to three identical or different C1-C4 hydroxyalkyl radicals, may be used. By way of example, organic amines such as monoethanolamine, diethanolamine, triethanolamine, monoisopropanolamine, diisopropanolamine, triisopropanolamine, N,N-dimethylethanolamine, 2-amino-2-methyl-1-propanol (AMP), 2-amino-2-methyl-1,3-propanediol, 3-amino-1,2-propanediol, 3-dimethylamino-1,2-propanediol, tris(hydroxymethyl) amino methane, guanidine, or combinations of two or more thereof may be chosen.

If present, the total amount of organic amine compounds may range from about 0.01% up to about 15%, such as up to about 14%, up to about 13%, up to about 12%, up to about 11%, up to about 10%, up to about 9%, up to about 8%, up to about 7%, up to about 6%, up to about 5%, up to about 4.5%, up to about 4%, up to about 3.5%, up to about 3%, up to about 2.5%, or up to about 2% by weight, relative to the total weight of the hair-color removing composition. For example, the total amount of organic amine compounds may range from about 0.01% to about 12%, from about 0.1% to about 10%, from about 0.5% to about 8%, or from about 1% to about 6% by weight, relative to the total weight of the composition. In at least some embodiments, the compositions comprise at least one organic amine compound, and have a total amount of organic amine compounds ranging from about 0.5% to about 12%, from about 0.5% to about 10%, from about 0.5% to about 8%, from about 0.5% to about 5%, from about 0.5% to about 4%, from about 0.5% to about 3%, from about 0.75% to about 12%, from about 0.75% to about 10%, from about 0.75% to about 8%, from about 0.75% to about 5%, from about 0.75% to about 4%, from about 0.75% to about 3%, from about 1% to about 12%, from about 1% to about 10%, from about 1% to about 8%, from about 1% to about 5%, from about 1% to about 4%, from about 1% to about 3%, from about 1.25% to about 12%, from about 1.25% to about 10%, from about 1.25% to about 8%, from about 1.25% to about 5%, from about 1.25% to about 4%, from about 1.25% to about 3%, from about 1.5% to about 12%, from about 1.5% to about 10%, from about 1.5% to about 8%, from about 1.5% to about 5%, from about 1.5% to about 4%, or from about 1.5% to about 3%, by weight relative to the total weight of the composition. In various embodiments, the total amount of organic amine compounds may be about 0.25%, about 0.5%, about 0.75%, about 1%, about 1.25%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 10.5%, about 11%, about 11.5%, or about 12% by weight, relative to the total weight of the composition, or may be present in any range using any of the foregoing as upper and lower limits.

### Auxiliary Components

Hair color-removing compositions that can be used in methods according to the disclosure may optionally include one or more auxiliary components. Non-limiting examples include preservatives, fragrances such as parfum, pH adjusters, salts, antioxidants, vitamins (e.g. ascorbic acid, tocopherol), vitamin derivatives, botanical extracts, buffers, sequestering agents, and the like. In some embodiments, the hair-color removing compositions are free or essentially free of any of the aforementioned, e.g. the compositions may be free or essentially free of antioxidants, free or essentially free of ascorbic acid, etc.

The total amount of auxiliary components, if present, typically ranges from about 0.01% to about 15% by weight, based on the total weight of the hair-color removing composition. For example, in some embodiments the individual amounts of each component or the total amount of components may range from about 0.1% to about 10%, about 0.1% to about 8%, about 0.1% to about 5%, about 0.1% to about 4%, about 0.1% to about 3%, about 0.1% to about 2%, about 0.25% to about 10%, about 0.25% to about 8%, about 0.25% to about 5%, about 0.25% to about 4%, about 0.25% to about 3%, about 0.25% to about 2%, about 0.5% to about 10%, about 0.5% to about 8%, about 0.5% to about 5%, about 0.5% to about 4%, about 0.5% to about 3%, about 0.5% to about 2%, about 0.75% to about 10%, about 0.75% to about 8%, about 0.75% to about 5%, about 0.75% to about 4%, about 0.75% to about 3%, or about 0.75% to about 2% by weight, based on the total weight of the composition.

In some embodiments, the hair-color removing compositions may be free or essentially free of one or more of the following: ascorbic acid and salts thereof, dyes or pigments for coloring the hair, persulfates, peroxides, and/or ammonia. In yet other embodiments, the compositions may comprise less than 5%, less than 4.5%, less than 4%, less than 3.5%, less than 3%, less than 2.5%, less than 2%, less than 1.5%, less than 1.25%, less than 1%, less than 0.75%, less than 0.5%, less than 0.4%, less than 0.3%, less than 0.2%, less than 0.1%, less than 0.05%, or less than 0.01% of a total amount of ascorbic acid and salts thereof, persulfates, peroxides, or ammonia, or of a total amount of any combination of two or more thereof.

The hair-color removing compositions may be in any suitable form. For example, the compositions may be a liquid, a gel, a gel cream, a cream, a serum, etc.

The pH of the hair-color removing composition is typically acidic, i.e. below 7. For example, compositions may have a pH ranging from about 1 to about 7, such as from about 2.5 to about 6.5. In some embodiments, the compositions have a pH ranging from about 2.5 to about 5, from about 2.75 to about 4.75, from about 3 to about 4.5, or from about 3 to about 4. For example, the pH of the composition may be about 3, about 3.25, about 3.5, about 3.75, about 4, about 4.25, or about 4.5.

### II. Kits

The disclosure also relates to kits. In various embodiments, the methods of removing color and/or altering the color, tone, and/or shade of hair comprise applying a pre- and/or post-treatment composition to the hair in connection with applying a hair color-removing composition to the hair. As such, a non-limiting example of a kit could include a first compartment or container comprising a pre- and/or post-treatment composition according to the disclosure, and a second compartment or container comprising a hair color-removing composition.

Optionally, kits according to the disclosure can include one or more additional containers or compartments, for example with shampoo compositions, conditioning compositions, and the like. Kits may also optionally include instructions, as well as implements for mixing and/or applying the compositions to the hair.

### III. Methods

In various embodiments, the disclosure relates to methods for altering the color, tone, and/or shade of hair. The compositions and methods can be used to remove some or all of certain dyes, while having minimal or no impact on other dyes. As such, the compositions can be targeted to remove certain dyes from the hair to achieve the consumer's desired result. For example, the methods may comprise methods of increasing the warmth of a hair color, for example by targeting dyes for removal that cause a color to be cooler. As a further example, the methods may comprise methods of increasing cool tones by targeting dyes for removal that cause a color to be warmer.

It is understood by those skilled in the art that the color, tone, and shade of hair are evaluated using the CIE L*a* b*system. In this system, the change in color, tone, and/or shade is determined by evaluating the color of hair after treatment (L*₂, a*₂, b*₂) compared to the color of the hair before treatment (L*₁, a*₁, b*₁). The overall change in color (ΔE) is defined as:

$$Δ {E}_{ab}^{*} = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}}$$

In this system, the three parameters represent, respectively, the color intensity (L*), the green/red color axis (a*) (for example, higher a*is more red) and the blue/yellow color axis (b*) (for example, higher b*is more yellow). The higher the value of L*, the lighter the color, the higher the value of a*, the redder the color, and the higher the value of b*, the yellower the color. Additionally, the higher the value for ΔE, the greater the difference in color of treated hair relative to the color of the hair prior to treatment. Thus, methods may comprise altering the color, tone, and/or shade of hair by varying L*₂, a*₂, and/or b*₂ values, and/or by increasing the ΔE value relative to the hair prior to treatment.

The compositions and methods are thus able to achieve a desired hair color, tone, and/or shade even without, or with minimal, application of, subsequent hair coloring compositions. However, although the hair color, tone, and/or shade can be modified using compositions and methods according to the disclosure, in at least some embodiments the methods comprise an additional step of coloring or toning the hair after the hair is treated according to the disclosure.

The disclosure also relates to methods of removing artificial color from keratin fibers, especially hair that has been previously dyed with an oxidation dye. In particular, the compositions and methods according to the disclosure are particularly useful for removing darker colors from hair that may be otherwise difficult to remove with traditional hair-color removing compositions that do not use oxidizing agents. By way of non-limiting example, darker colors that can be removed in methods according to the disclosure include blacks, ash browns, browns, ash mochas, natural ash (blue or green), ash violets, violets, red violets, reds, red mochas, red browns, mahogany, red coppers, coppers, and golds.

Methods according to the disclosure comprise using treatment compositions according to the disclosure before, during, or after a hair color-removing composition has been applied to the hair. Thus, the methods may be methods of pre-treating the hair, methods of post-treating the hair, and/or methods of treating the hair during a color-removing process.

Exemplary pre-treatment methods comprise (i) applying a treatment composition according to the disclosure onto keratin fibers, e.g. hair that has been previously-dyed with at least one oxidative dye compound, and optionally leaving the treatment composition on the keratin fibers for a period of time (“processing time” or “leave-in period”), and (ii) subsequently applying a hair color-removing composition onto the keratin fibers, and optionally leaving the hair color-removing composition on the keratin fibers for a period of time. The hair may be rinsed and/or shampooed after step (ii), but preferably, the hair is not rinsed and/or shampooed between steps (i) and (ii).

Exemplary post-treatment methods comprise (i) applying a hair color-removing composition onto keratin fibers, e.g. hair that has been previously-dyed with at least one oxidative dye compound, and optionally leaving the hair color-removing composition on the keratin fibers for a period of time, and (ii) applying a treatment composition according to the disclosure onto the keratin fibers, and optionally leaving the treatment composition on the keratin fibers for a period of time. The hair may be rinsed and/or shampooed after step (ii), but preferably, the hair is not rinsed and/or shampooed between steps (i) and (ii).

Further exemplary methods comprising using the treatment compositions as both a pre-treatment and post-treatment. For example, such a method may comprise (i) applying a treatment composition according to the disclosure onto keratin fibers, optionally leaving the treatment composition on the keratin fibers for a period of time, (ii) subsequently applying a hair color-removing composition onto the keratin fibers, optionally leaving the hair color-removing composition on the keratin fibers for a period of time, and (iii) subsequently applying a treatment composition according to the disclosure onto the keratin fibers, optionally leaving the treatment composition on the keratin fibers for a period of time. The hair may be rinsed and/or shampooed after step (iii), but preferably, the hair is not rinsed and/or shampooed between steps (i), (ii), and/or (iii). It should be understood that in such embodiments, the pre- and post-treatment compositions may be the same or different.

Still further exemplary methods comprise applying a treatment composition according to the disclosure onto the keratin fibers substantially simultaneously with application of a hair color-removing composition to the keratin fibers. For example, this may include a layering of treatment composition and hair color-removing composition onto the hair.

The appropriate leave-in period for both the treatment composition and hair color-removing composition will be determined based on the color to be removed, and may in various embodiments last up to about 5 minutes, up to about 10 minutes, up to about 20 minutes, up to about 30 minutes, up to about 45 minutes, up to about 1 hour, up to about 2 hours, etc., such as from about 1 minute to about 60 minutes, from about 2 minutes to about 50 minutes, from about 5 minutes to about 45 minutes, or from about 10 minutes to about 40 minutes. It may be advantageous, in various embodiments, to leave the treatment and/or hair color-removing composition on the hair for at least about 15 minutes, and up to about 60 minutes. For example, the leave-in period may range from about 15 minutes to about 60 minutes, from about 20 minutes to about 60 minutes, or from about 25 minutes to about 60 minutes. Optionally, the hair may be covered, such as with a processing cap or aluminum foil, for at least a portion of any of the leave-in periods.

Optionally, the hair to which a treatment and/or hair color-removing composition according to the disclosure has been applied may be heated during the leave-in period. For example, the hair may be heated using a hair dryer, hood dryer, or any other method for at least portion of the leave-in period. The hair may be heated using temperatures above room temperature, for example ranging from about 27° C. to about 90° C., from about 30° C. to about 80° C., from about 35° C. to about 70° C., or from about 40° C. to about 50° C. For example, an exemplary heating temperature range may be about 40° C. to about 45° C., such as about 40° C. to about 43° C.

In various embodiments, it may be advantageous to heat the treated hair for at least a portion of the leave-in period, with no heating for at least a portion of the leave-in period. By way of example, after the treatment and/or hair color-removing composition is applied to the hair, the treated hair may be left to process for a period of time at room temperature, followed by a period of time with application of heat. As an alternate example, after the treatment and/or hair color-removing composition is applied to the hair, the treated hair may be heated, followed by a period of time when the hair processes at room temperature. Further variations on such methods of alternating heating the treated hair and allowing the treated hair to process at room temperature may be used, such as, for example, heating followed by room temperature followed by heating, or processing at room temperature followed by heating followed by processing at room temperature, and so on.

Surprisingly, the compositions and methods according to the disclosure effectively alter the color of the hair and/or remove color from hair without the use of oxidizing agents, persulfates, peroxides, and/or ammonia, and without the damage that is typically associated with color removing compositions containing these components.

It should be understood that treatment and hair color-altering compositions according to the disclosure are not compositions for coloring the hair. As such, the compositions are free or essentially free of dyes or pigments for coloring the hair, in particular of oxidative dye compounds. The compositions may optionally comprise a dye or pigment for coloring the composition, but it will be understood that the amount and/or type of dye or pigment will be chosen so that it does not impart color to the hair. Likewise, the methods according to the disclosure are not methods for imparting color to the hair.

Having described the many embodiments of the present invention in detail, it will be apparent that modifications and variations are possible without departing from the scope of the disclosure defined in the appended claims. Furthermore, it should be appreciated that all examples in the present disclosure, while illustrating many embodiments of the disclosure, are provided as non-limiting examples and are, therefore, not to be taken as limiting the various aspects so illustrated. It is to be understood that all definitions herein are provided for the present disclosure only.

As used herein, the terms “comprising,” “having,” and “including” (or “comprise,” “have,” and “include”) are used in their open, non-limiting sense.

In this application, the use of the singular includes the plural unless specifically stated otherwise. The singular forms “a,” “an,” “the,” and “at least one” are understood to encompass the plural as well as the singular unless the context clearly dictates otherwise. The expression “one or more” and “at least one” are interchangeable and expressly include individual components as well as mixtures/combinations. Likewise, the term “a salt thereof” also relates to “salts thereof.” Thus, where the disclosure refers to “at least one element selected from the group consisting of A, B, C, D, E, F, a salt thereof, or mixtures thereof,” it indicates that that one or more of A, B, C, D, and F may be included, one or more of a salt of A, a salt of B, a salt of C, a salt of D, a salt of E, and a salt of F may be included, or a mixture of any two or more of A, B, C, D, E, F, one or more salts of A, one or more salts of B, one or more salts of C, one or more salts of D, one or more salts of E, and one or more salts of F may be included.

The term “and/or” should be understood to include both the conjunctive and the disjunctive. For example, “water and/or non-aqueous solvents” means “water and non-aqueous solvents” as well as “water or non-aqueous solvents,” and expressly covers instances of either.

As used herein, the phrases “and mixtures thereof,” “and a mixture thereof,” “and combinations thereof,” “and a combination thereof,” “or mixtures thereof,” “or a mixture thereof,” “or combinations thereof,” and “or a combination thereof,” are used interchangeably to denote that the listing of components immediately preceding the phrase, such as “A, B, C, D, or mixtures thereof” signify that the component(s) may be chosen from A, from B, from C, from D, from A+B, from A+B+C, from A+D, from A+C+D, etc., without limitation on the variations thereof. Thus, the components may be used individually or in any combination thereof.

For purposes of the present disclosure, it should be noted that to provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

All ranges and amounts given herein are intended to include sub-ranges and amounts using any disclosed point as an end point, and all endpoints are intended to be included unless expressly stated otherwise. Thus, a range of “1% to 10%, such as 2% to 8%, such as 3% to 5%,” is intended to encompass ranges of “1% to 8%,” “1% to 5%,” “2% to 10%,” and so on. All numbers, amounts, ranges, etc., are intended to be modified by the term “about,” whether or not expressly stated, unless expressly stated otherwise. Similarly, a range given of “about 1% to 10%” is intended to have the term “about” modifying both the 1% and the 10% endpoints. The term “about” is used herein to indicate a difference of up to +/−10% from the stated number, such as +/−9%, +/−8%, +/−7%, +/−6%, +/−5%, +/−4%, +/−3%, +/−2%, or +/−1%. Unless expressly stated otherwise, “about” means +/−5%. Likewise, all endpoints of ranges are understood to be individually disclosed, such that, for example, a range of 1:2 to 2:1 is understood to disclose a ratio of both 1:2 and 2:1.

As used herein, if a component is described as being present “in an amount up to” a certain amount, it is intended that such component is, in fact, present in the composition, i.e. is present in an amount greater than 0%.

All amounts and ratios herein are given based upon the total weight of the composition, unless otherwise indicated. Unless otherwise indicated, all percentages herein are by weight of active material.

As used herein, the phrase “applying a composition onto keratin fibers” and variations thereof are intended to mean contacting the keratin fibers such as hair with at least one of the compositions of the disclosure, in any manner. It may also mean contacting the keratin fibers with an effective amount of the composition.

As used herein, the term “salts” referred to throughout the disclosure may include salts having a counterion such as an alkali metal, alkaline earth metal, or ammonium counterion. This list of counterions, however, is non-limiting. Salts also include a dissociated form of a compound, e.g. in an aqueous solution.

Unless otherwise defined for any specific embodiment, the term “substantially free” or “essentially free” as used herein means that there is less than about 5% by weight of a specific material added to a composition, based on the total weight of the composition. For example, the compositions may include less than about 4%, less than about 3%, less than about 2%, less than about 1.5%, less than about 1%, less than about 0.5%, less than about 0.1%, less than about 0.01%, less than about 0.001%, or less than about 0.0001% of the specified material. As such, it is contemplated that any component described herein for use in the compositions can be present in the compositions in amounts less than about 5%, less than about 4%, less than about 3%, less than about 2%, less than about 1%, less than about 0.5%, less than about 0.1%, less than about 0.01%, less than about 0.001%, or less than about 0.0001%, and the composition will be considered “substantially free” of such material. A composition that is “free” of a component is understood to contain none of the specified component. However, it is understood that the terms “free” and “substantially free” refer to the amount of a component added to the composition, without including an amount of the component present in the composition as a minor component in a raw material. For example, a composition that is “free” of waxes may not have wax included as an intended component but may nevertheless contain a pigment that is coated with a wax, as such wax would be considered a minor component of the pigment material and would not be expected to provide benefits to the composition that would be expected by including a wax per se as an intended component.

As used herein, the terms “treat,” “treated,” “treatment,” and variations thereof is not intended to be limiting, but rather is merely intended to indicate that one or more compositions is applied to the hair, and optionally removed from the hair. For example, hair that is “treated” with a composition according to the disclosure may have had the composition applied, and/or may have had the composition applied and removed, e.g. by rinsing or towel drying. As a further example, hair that is “treated” with a composition according to the disclosure may have had the composition applied, and/or may have had the composition applied and rinsed from the hair.

As used herein, “altering the color of hair,” “modifying the color of hair,” and variations thereof means that the color, tone, and/or shade of hair is changed by removing previously-applied hair dyes from the hair. Further, the terms “color removal,” “hair color-removing,” and variations thereof should be understood to refer to removal of some or all of previously-applied hair dyes from the hair. It should be understood that removal of artificial color can include removal of all color, or can include removal of various tones of color. Therefore, in some embodiments, overall change in the hair color will be observed (e.g. greater ΔE), while in other embodiments even if the overall color change is not considered significant, variation in one or more of the L*, a*, or b*values will be observed.

As used herein, “artificial color” means color that has been imparted to the hair by means of prior application of a composition capable of changing the color of hair, for example with permanent, semi-permanent, or demi-permanent hair dye compositions. In various embodiments the artificial color that is removed from the hair is color that has been imparted to the hair by one or more oxidative dye compounds.

As used herein, “thiol-based” is used according to its ordinary meaning to signify that the compound has an R-SH (thiol) group, where R is an alkyl group or other organic substituent.

The examples that follow serve to illustrate embodiments of the present disclosure without, however, being limiting in nature. It will be apparent to those skilled in the art that various modifications and variations can be made in the compositions and methods of the invention without departing from the spirit or scope of the invention.

### EXAMPLES

The following Examples are intended to be non-limiting and explanatory in nature only. In the Examples, amounts are expressed in percentage by weight (wt %) of active materials, relative to the total weight of the composition, unless otherwise indicated.

In these Examples, the change in the color of hair is evaluated with the CIE L*a* b*system using Colorshot MS, where the change is determined by evaluating the color of the hair after treatment (L*₂, a*₂, b*₂) compared to the color of the hair before treatment (L*₁, a*₁, b*₁). The change in color (ΔE) is calculated as follows:

$$Δ {E}_{ab}^{*} = \sqrt{{( {L}_{2}^{*} - {L}_{1}^{*} )}^{2} + {( {a}_{2}^{*} - {a}_{1}^{*} )}^{2} + {( {b}_{2}^{*} - {b}_{1}^{*} )}^{2}} .$$

### Example 1-Compositions

Treatment compositions 1A-1B according to the disclosure, and hair color-removing compositions R1-R2, were prepared as shown in Table 1A. Comparative compositions C1-C4 were prepared as shown in Table 1B.

TABLE 1A

| | 1A | 1B | R1 | R2 |
|---|---|---|---|---|
| XANTHAN GUM | | | 0.7 | 0.7 |
| BENTONITE | | | 5.0 | |
| ETHANOLAMINE | 10.0 | | 1.7 | 3.3 |
| AMINOMETHYL | | 10.0 | | |
| PROPANOL | | | | |
| KETOGLUTARIC | | | 10.0 | 10.0 |
| ACID | | | | |
| ARGININE | | | 3.0 | 3.0 |
| THIOSALYCYLIC | | | | 5.0 |
| ACID | | | | |
| THIOLACTIC ACID | | | 5.0 | |
| SODIUM LAURYL | | | 1.5 | 1.5 |
| SULFATE | | | | |
| FRAGRANCE | | | 1.0 | 1.0 |
| SOLVENTS | QS to 100 | QS to 100 | QS to 100 | QS to 100 |

TABLE 1B

| | C1 | C2 | C3 | C4 |
|---|---|---|---|---|
| BENTONITE AND/OR KAOLIN | 5.0 | | | 0.05 |
| ETHANOLAMINE | 11.7 | | | |
| KETOGLUTARIC ACID | 10.0 | | | |
| ARGININE | 3.0 | | | |
| THIOLACTIC ACID | 5.0 | | | |
| TRIDECETH-2 CARBOXAMIDE | | 0.9 | | |
| MEA | | | | |
| TETRASODIUM ETIDRONATE | | 0.06 | | |
| SODIUM SALICYLATE | | 0.04 | | |
| GLYCERIN | | 0.5 | | |
| CETEARYL ALCOHOL | | 2.3 | | |
| CETEARETH-25 | | 0.6 | | |
| HYDROGEN PEROXIDE | | 6.0 | | |
| TETRASODIUM PYROPHOSPHATE | | 0.04 | | |
| AMMONIA | | | 10.0 | |
| AMMONIUM CHLORIDE | | | | 5.0 |
| STRONTIUM PEROXIDE | | | | 11.5 |
| SODIUM BENZOATE | | | | 0.3 |
| DIETHYLHEXYL SODIUM | | | | 1.7 |
| SULFOSUCCINATE | | | | |
| EDTA | | | | 1.0 |
| SODIUM METASILICATE | | | | 9.0 |
| SODIUM LAURYL SULFATE | 1.5 | | | 14.0 |
| POTASSIUM PERSULFATE | | | | 20.0 |
| SODIUM PERSULFATE | | | | 33.0 |
| CELLULOSE AND/OR XANTHAN | 0.7 | | | 4.0 |
| GUM | | | | |
| WATER | QS to | QS to | QS to | QS to |
| | 100 | 100 | 100 | 100 |

### Example 2-Evaluation of Color Removal/Hair Color Alteration

The following studies were performed to evaluate the efficacy of compositions according to the disclosure to remove color and to alter the color, tone, and/or shade of hair that has previously been dyed with oxidative hair dye compositions.

Studies using three different commercially-available oxidative hair color compositions, each having different combinations of oxidative dye compounds, were performed as follows. Swatches of permed, ˜90% grey hair were colored by mixing the dye composition, in a 1:1 ratio, with 20V hydrogen peroxide developer to form a hair dye mixture, and the hair dye mixture was applied to the swatches at a rate of about 3 grams per gram of hair. The swatches were left to process at 27° C. for about 35 minutes, rinsed, then shampooed with a commercial shampoo and rinsed again. The swatches were then subjected to four additional shampoo/rinse cycles, and dried. Once the swatches were dry, all but one swatch in each color study was treated as follows. One swatch per color had no color-removing composition applied, and was the color control for that study.

One-step process: One of compositions 1A-1B, R1-R2, C₁, or C4 was applied to separate dyed swatches at a rate of about 2 grams per gram of hair. After the swatches were allowed to process for 30 minutes at about 33° C., the swatches were rinsed for about 30 seconds, shampooed with a commercial shampoo, rinsed, shampooed again, rinsed again, and then dried.

One-step, repeated application: The one-step process was followed with compositions R1 or R2, but with a second application of the hair-color removing composition after the one-step process was completed.

Two-step process: One of treatment compositions 1A or 1B, or one of comparative compositions C2, C3, or C4 was applied to separate dyed swatches either before or after hair color-removing compositions R1 or R2. In each case, the hair color-removing compositions and the treatment and comparative compositions were allowed to process for 30 minutes at about 33° C. When treatment compositions 1A or 1B, or comparative compositions C2, C3, or C4 was used as a pre-treatment, the swatches were not rinsed before application of R1 or R2; however, after the processing time for R1 or R2 was complete the swatches were rinsed for about 30 seconds, shampooed with a commercial shampoo, rinsed, shampooed again, rinsed again, and then dried. When treatment compositions 1A or 1B, or comparative compositions C2, C3, or C4 was used as a post-treatment, the swatches were not rinsed after application of R1 or R2, but after the processing time for treatment compositions 1A or 1B, or comparative compositions C2, C3, or C4 was complete the swatches were rinsed for about 30 seconds, shampooed with a commercial shampoo, rinsed, shampooed again, rinsed again, and then dried.

After the above processes were complete and the swatches were dry, the color of each of the swatches was evaluated with the CIE L*a* b*system. Tables 2A-2C show the ΔE values for the treated swatches, relative to the color control, and the L*, a*, and b*results for each study.

TABLE 2A
Results: Color Study 1

| | Study | | Study 1 | | | |
|---|---|---|---|---|---|---|
| Process | No. | Composition(s) | L* | a* | b* | ΔE |
| One-step | 1-1 | R1 | 39.1 | 15.9 | 26.4 | 44.6 |
| | 1-2 | 1B | No change | | | |
| | 1-3 | C1 | 6.3 | 2.7 | −3 | 3.43 |
| | 1-4 | R2 | 42.5 | 8 | 28.8 | 46.9 |
| | 1-5 | 1A | No change | | | |
| One-step, | 1-6 | R1 × 2 | 46.4 | 11.3 | 21.7 | 46.9 |
| repeated | 1-7 | R2 × 2 | 44.5 | 8.1 | 29.2 | 48.67 |
| Two-step | 1-8 | 1B pre-treatment | 22.7 | 12 | 12.5 | 23.25 |
| | | followed by R1 | | | | |
| | 1-9 | R1 followed by | 45 | 5.3 | 25 | 46.5 |
| | | 1B post-treatment | | | | |
| | 1-10 | 1B pre-treatment | 29.2 | 13.8 | 19.1 | 32.39 |
| | | followed by R2 | | | | |
| | 1-11 | R2 followed by | 45.1 | 6.5 | 24.6 | 46.42 |
| | | 1B post-treatment | | | | |
| | 1-12 | R2 followed by | 39.3 | 6.7 | 24.7 | 41.71 |
| | | 1A post-treatment | | | | |

TABLE 2B
Results: Color Study 2

| | Study | | Study 2 | | | |
|---|---|---|---|---|---|---|
| Process | No. | Composition(s) | L* | a* | b* | ΔE |
| One-step | 2-1 | R1 | 12.7 | 19.7 | 10.9 | 14.2 |
| | 2-2 | R2 | 9.6 | 13.9 | 7.5 | 6.96 |
| | 2-3 | 1B | No change | | | |
| | 2-4 | C1 | 11.2 | 17 | 8.3 | 10.29 |
| | 2-5 | C4 | 54.6 | 27.2 | 58.8 | 74.11 |
| | 2-6 | 1A | No change | | | |
| One-step, | 2-7 | R1 × 2 | 14.3 | 21.5 | 12.8 | 17 |
| repeated | 2-8 | R2 × 2 | 14.2 | 23 | 14.1 | 18.9 |
| Two-step | 2-9 | 1B pre-treatment | 17.3 | 26.2 | 16.6 | 23.7 |
| | | followed by R1 | | | | |
| | 2-10 | R1 followed by | 15.2 | 20.9 | 13.6 | 17.4 |
| | | 1B post-treatment | | | | |
| | 2-11 | C2 pre-treatment | 16.2 | 23.8 | 15.5 | 20.9 |
| | | followed by R1 | | | | |
| | 2-12 | R1 followed by | 11.8 | 15 | 8.3 | 8.7 |
| | | C2 post-treatment | | | | |
| | 2-13 | 1B pre-treatment | 10.8 | 16 | 8.1 | 9.27 |
| | | followed by R2 | | | | |
| | 2-14 | R2 followed by | 13.9 | 21.2 | 12.6 | 16.57 |
| | | 1B post-treatment | | | | |
| | 2-15 | R2 followed by | 11.7 | 18.7 | 9.3 | 12.3 |
| | | C3 post-treatment | | | | |
| | 2-16 | R2 followed by | 47.1 | 28 | 56.3 | 67.99 |
| | | C4 post-treatment | | | | |
| | 2-17 | R2 followed by | 13.6 | 22.4 | 11.9 | 17.15 |
| | | 1A post-treatment | | | | |

TABLE 2C
Results: Color Study 3

| | Study | | Study 3 | | | |
|---|---|---|---|---|---|---|
| Process | No. | Composition(s) | L* | a* | b* | ΔE |
| One-step | 3-1 | R1 | 45.9 | 20.9 | 20 | 32.9 |
| | 3-2 | R2 | 47.3 | 17.8 | 20.13 | 35.55 |
| | 3-3 | C1 | 36.1 | 28.4 | 21 | 21.37 |
| | 3-4 | 1A | No change | | | |
| | 3-5 | 1B | No change | | | |
| One-step, | 3-6 | R1 × 2 | 54.7 | 11.9 | 18.7 | 44.7 |
| repeated | 3-7 | R2 × 2 | 56.1 | 12.7 | 20.3 | 45.69 |
| Two-step | 3-8 | 1B pre-treatment | 46 | 20 | 17.6 | 35.74 |
| | | followed by R1 | | | | |
| | 3-9 | R1 followed by | 47.3 | 17 | 19.2 | 33.24 |
| | | 1B post-treatment | | | | |
| | 3-10 | 1B pre-treatment | 52.6 | 11.7 | 21.7 | 43.22 |
| | | followed by R2 | | | | |
| | 3-11 | R2 followed by | 46.6 | 19.3 | 15.1 | 34.07 |
| | | 1B post-treatment | | | | |
| | 3-12 | R2 followed by | 47.1 | 16.4 | 15.7 | 35.81 |
| | | 1A post-treatment | | | | |

The results in Tables 2A-2C demonstrate that using treatment compositions according to the disclosure results in greater overall color change (greater ΔE). These results are surprising because they show that using the treatment compositions as pre- and/or post-treatment compositions has a greater ability to remove color from the hair than including the same amount of organic amine compound in the hair color-removing composition, as a comparison of study 1-3 with studies 1-8 and 1-9, a comparison of study 2-4 with studies 2-10 and 2-11, and a comparison of study 3-4 with studies 3-9 and 3-10 show. These results are also surprising because they demonstrate that using the treatment compositions as pre- and/or post-treatment compositions has a greater ability to remove color from the hair than using other pre- and/or post-treatment compositions such as an oxidizing composition, as a comparison of study 2-9 with study 2-11 and a comparison of study 2-10 with study 2-12 show.

In addition, the results show that the color, tone, and/or shade of hair can be altered in a controlled manner using compositions and methods according to the disclosure. For example, as Study 3 shows, relative to the hair color-removing composition study 3-2 which provides approximately the same overall change in color (ΔE), the process of study 3-8 provides a more reddish and less yellow tone (higher a*value and lower b*value).

These data demonstrate that the compositions and processes according to the disclosure can be used to modify hair color in a targeted manner, unlike traditional hair color-removing compositions. Compositions and processes according to the disclosure have the unique ability to fade-on-tone and deliver the desired color, tone, and/or shade, even in the absence of subsequent hair color applications.

### Example 3-Additional Treatment Compositions

Treatment compositions 2A-2D can also be prepared as shown in Table 3, and are likewise expected to provide similar hair color removing and/or modification benefits when used in methods according to the disclosure.

TABLE 3

| | 2A | 2B | 2C | 2D |
|---|---|---|---|---|
| ARGININE | | 3.0 | | 1.0 |
| XANTHAN GUM | 0.7 | 0.7 | | |
| HYDROXYPROPYL | | | 1.5 | 1.0 |
| CELLULOSE | | | | |
| TRIETHANOLAMINE | 10.0 | | | |
| ETHANOLAMINE | | 16.0 | 7.5 | |
| AMINOMETHYL | | | | 4.0 |
| PROPANOL | | | | |
| FATTY COMPOUNDS | 2.0 | | 0.5 | |
| (silicone oils or | | | | |
| plant oils) | | | | |
| ADDITIVES | ≤5 | ≤5 | ≤5 | ≤5 |
| (fragrances, pH | | | | |
| adjuster, vitamins, plant | | | | |
| extracts, preservatives, | | | | |
| etc.) | | | | |
| SOLVENTS (water | QS to 100 | QS to 100 | QS to 100 | QS to 100 |
| and non- | | | | |
| aqueous solvents) | | | | |

The above Examples demonstrate that compositions and methods according to the disclosure effectively remove various oxidative hair dyes from hair, allowing the hair color, tone, and/or shade to be altered in a controlled manner.

These compositions and processes are unique in that they can provide a targeted method for removing hair color to provide consumers with choices in resulting color, even without the need to backfill the color to arrive at the desired result. The compositions and processes can be particularly useful when similar tonality, rather than an entirely different hair color, is desired, as the data in the above Examples demonstrate.

Moreover, the Examples show that the compositions are processes are particularly useful for removing darker colors, which tend to be more difficult to remove with color-removing agents that are not oxidizing agents.

1. A method for altering the color of hair, comprising:
(i) applying a treatment composition to the hair, the treatment composition comprising:
a) at least one organic amine compound;
b) at least one solvent; and
c) optionally at least one additional component chosen from surfactants, fatty compounds, thickening agents, amino acids and/or salts thereof, amino-sulfonic acids and/or salts thereof, or combinations of two or more thereof, and
(ii) applying a hair color-removing composition to the hair,
wherein step (i) occurs before, during, and/or after step (ii).

2. The method according to claim 1, wherein the hair color-removing composition comprises at least one hair color-removing agent chosen from thiolactic acid, thiosalicylic acid, keto-glutaric acid, and/or salts thereof.

3. The method according to claim 1, wherein the total amount of organic amines present in the treatment composition ranges from about 5% to about 20% by weight, relative to the total weight of the treatment composition.

4. The method according to claim 1, wherein the treatment composition comprises ethanolamine and/or aminomethyl propanol.

5. The method according to claim 1, wherein the pH of the treatment composition ranges from about 9 to about 13.

6. The method according to claim 1, wherein the treatment composition and/or the hair color-removing composition is left on the hair for a leave-in period ranging from about 5 minutes to about 60 minutes.

7. The method according to claim 6, wherein the hair is heated during the leave-in period.

8. The method according to claim 1, wherein step (i) occurs before and/or after step (ii).

9. The method according to claim 1, which is a method for removing oxidative color from the hair.

10. A method for altering the color of hair, comprising:
(i) applying a treatment composition comprising water and from about 5% to about 15% of one or more organic amines to the hair; and
(ii) applying a hair color-removing composition comprising at least one thiol-based color-removing compound to the hair,
wherein step (i) occurs before and/or after step (ii).

11. The method according to claim 10, wherein the treatment composition comprises ethanolamine and/or aminomethyl propanol.

12. The method according to claim 10, wherein the hair color-removing composition further comprises keto-glutaric acid and/or a salt thereof.

13. The method according to claim 10, wherein the pH of the treatment composition ranges from about 9 to about 13.

14. The method according to claim 10, wherein the treatment composition and/or the hair color-removing composition is left on the hair for a leave-in period ranging from about 5 minutes to about 60 minutes.

15. The method according to claim 14, wherein the hair is heated during the leave-in period.

16. A kit comprising:
(i) a first compartment or container comprising a treatment composition, the treatment composition comprising:
a) at least one organic amine;
b) at least one solvent; and
c) optionally at least one additional component chosen from surfactants, fatty compounds, thickening agents, amino acids and/or salts thereof, amino-sulfonic acids and/or salts thereof, or combinations of two or more thereof, and
(ii) a second compartment or container comprising a hair color-removing composition.

17. The kit according to claim 16, wherein the treatment composition comprises ethanolamine and/or aminomethyl propanol.

18. The kit according to claim 16, wherein the total amount of organic amines present in the treatment composition ranges from about 5% to about 20% by weight, relative to the total weight of the treatment composition.

19. The kit according to claim 16, wherein the pH of the treatment composition ranges from about 9 to about 13.

20. The kit according to claim 16, wherein the hair color-removing composition comprises at least one hair color-removing agent chosen from thiolactic acid, thiosalicylic acid, keto-glutaric acid, and/or salts thereof.
